(19) United States
(12) Patent Application Publication
(10) Pub. No.: US 2006/0124459 A1
(43) Pub. Date: Jun. 15, 2006

(54) Devices and methods for focusing analytes in an electric field gradient II

(75) Inventors: David Strand, Sherborn, MA (US); Dan M. Leatzow, Butte, MI (US)

Correspondence Address: BANNER & WITCOFF, LTD., 28 STATE STREET, 28th FLOOR, BOSTON, MA, 02109-9601, US

(73) Assignee: Protassis Corporation, Marlborough (MA)

(21) Appl. No.: 10/542,509

(22) PCT Filed: Jan. 12, 2004
(86) PCT No.: PCT/US04/00659

§ 371 Date: Jan. 6, 2006

Related U.S. Application Data
(60) Provisional application No. 60/440,150, filed on Jan. 15, 2003.
(60) Provisional application No. 60/471,597, filed on May 19, 2003.

(51) Int. Cl. C07K 1/26; G01N 27/447

(52) U.S. Cl. 204/450; 204/600

(57) ABSTRACT

Devices are provided for separating and focusing analytes, comprising a separation chamber and electrodes separated from the separation chamber by a membrane. The electrodes are operative to generate an electric field in the separation chamber. Molecular sieve in the separation chamber is operative to shift the location at which a stationary focused band of the analyte forms under a given set of focusing process parameters. Methods are provided for separating and focusing charged analytes, comprising introducing a first fluid comprising at least one charged analyte into the separation chamber of a device as just described, applying an electric field gradient to the separation chamber to focus the charged analyte at a location in the separation chamber. Methods are provided for separating and focusing un-charged (including inadequately charged) analytes, comprising introducing a fluid comprising at least the uncharged analyte and lipids, micelles and/or vesicles into the separation chamber of a device as just described, and applying an electric field gradient to the separation chamber to focus the analyte (in association with the lipids, micelles and/or vesicles) at a location in the separation chamber.

### FIELD OF THE INVENTION

The present invention relates to electrophoretic devices and methods and, more particularly, to electrophoretic devices and methods that focus charged analytes in the presence of an electric field gradient.

### BACKGROUND OF THE INVENTION

Dynamic field gradient focusing (DFGF), as described in U.S. Pat. No. 6,277,258, incorporated herein in its entirety for all purposes, is capable of focusing certain charged analytes. In particular, DFGF is well adapted to concentrate an analyte, i.e., a target analyte or species, from a dilute fluid sample, in certain cases being able to separate one or more such analytes from other species present in the fluid sample by concentrating them at different locations in the DFGF chamber. Such focusing takes advantage of the target analyte's charge to mass ratio or electrophoretic mobility as the fluid sample is passed through an electric field gradient in a DFGF chamber. Thus, DFGF can be used to concentrate certain charged analytes, for example a desired protein, either on a batch-by-batch basis or in a continuous fashion. In addition, DFGF can be used in some cases to separate a desired analyte from other analytes in the fluid sample, by concentrating the desired analyte at a location in the DFGF chamber at which the other analytes do not concentrate under the conditions used, including the sample flow rate, field strength and gradient, properties of the carrier fluid such as pH, chamber and electrode configuration, etc.

In certain applications, however, DFGF is unable to adequately focus a desired analyte separately from other species in the fluid sample. For example, known DFGF techniques have not been shown to be well adapted to separating certain biological molecules, in particular biomacromolecules, from each other where such biological molecules have the same or similar charge to mass ratios or electrophoretic mobilities. Thus, such DFGF techniques have been inadequate for separating and focusing certain proteins, DNA and RNA molecules, and other large molecules from fluid samples containing other such species. The ability to focus and separate such molecules is desirable, for example, in drug discovery and other pharmaceutical research, pharmaceutical and other chemical testing and production processes and the like.

It is an object of the present invention to address one or more of the above-mentioned research and industrial needs. It is an object of at least certain preferred embodiments of the invention to provide devices and methods for focusing charged analytes, i.e., for concentrating such charged analytes and, as necessary, separating such charged analytes from other species in a fluid sample, including charged analytes that have the same or similar charge to mass ratios or electrophoretic mobilities. From the following summary and the detailed description of certain preferred embodiments, additional objects of the invention and objects of certain preferred embodiments of the invention will be apparent to those skilled in the art, i.e., to those having skill and experience in this area of technology.

### SUMMARY

In accordance with a first aspect, electrophoresis devices are provided for focusing a charged analyte. The electrophoretic devices comprise a separation chamber through which a sample fluid flows. An electric field gradient is established in the separation chamber such that a charged analyte flowed into the chamber will be subject to an electrophoretic force that opposes the hydrodynamic force of the flowing fluid. The magnitude of the electrophoretic force is determined by the net or apparent charge of the analyte and by its location within the electric field gradient, while the hydrodynamic force is determined by the hydrodynamic radius of the analyte and by the viscosity and speed of the flowing fluid. At some particular point in the separation chamber, the hydrodynamic force and the opposing electrophoretic forced will balance out and the analyte will be substantially held at that point, focusing into a band of charged analyte. The separation chamber of the device comprises molecular sieve. The molecular sieve is operative to shift the location at which a stationary focused band of a charged analyte forms under a given set of focusing process parameters. The molecular sieve may be fixed or soluble. Fixed molecular sieve may occupy any suitable portion of the volume of the separation chamber, preferably substantially the entire volume of the chamber. Soluble molecular sieve preferably is incorporated into a fluid sample containing the target analyte to be focused. Materials suitable for use as the molecular sieve in a device as disclosed here are discussed further below and, in general, are operative in conjunction with the other components of the device, for the intended analyte(s), under a suitable set of focusing process parameters, to shift the location at which a charged analyte is focused and held in the separation chamber as a function of the size or molecular weight of the analyte.

The separation chamber is generally an elongate chamber, typically having an inlet for introducing fluid into the separation chamber, and an outlet for exiting fluid from the chamber. These outlets are typically located proximate opposing longitudinal ends of the separation chamber. The electric field gradient will be present in the separation chamber for at least a portion of the chamber located between the inlet and outlet, optionally extending for the fill distance between the inlet and the outlet and may even extend beyond the inlet and/or the outlet. The electric field gradient will be such that there is a gradient longitudinally through the separation chamber, that is, so that an electric field gradient will exist along the direction of fluid flow in the separation chamber. In certain preferred embodiments, one or more additional ports are provided in the separation chamber for adding or removing fluid from the chamber, e.g., for drawing off fluid containing a focused analyte.

In certain preferred embodiments, the electrophoretic devices comprise a separation chamber for receiving a fluid containing one or more analytes. An electrode chamber is separated from the separation chamber by a porous, conductive membrane, which in certain preferred embodiments is substantially planar. In those embodiments described here that comprise a porous membrane, the membrane is at least conductive in that it does not prevent the electric field in the chamber and it is porous in the sense that it is permeable to buffer species or the like without allowing contact of the target analyte with the electrodes. In certain embodiments, the membrane does not substantially affect the electric field generated by the electrodes and does not affect the electric field experienced by the separation chamber. Electrodes are positioned proximate the electrode chamber and are operative to generate an electric field in the electrode channel. The separation chamber comprises molecular sieve. The electrode chamber is non-uniform. As used herein, “non-uniform” refers to a chamber that has a non-uniform cross-section, that is to say, the cross-sectional area of the chamber varies axially along the length of the chamber, length referring to the direction in which fluid flows through the separation chamber. For a non-uniform electrode chamber, this will mean that the electrode chamber has a cross-section that varies axially along the length of the chamber in a direction parallel to the flow of fluid through the separation chamber. Such a chamber is referred to at times herein as “configured.” The electrodes generate an electric field in the electrode chamber, where the non-uniformity of the electrode chamber establishes a gradient in the electric field. The electric field gradient is communicated to the separation chamber. The electrode chamber in certain preferred embodiments has a substantially uniform depth (depth here meaning the direction normal to the plane of the membrane) and a non-uniform or non-constant width (width here meaning the direction perpendicular to the overall direction of flow and parallel to the plane of the membrane). In other preferred embodiments, the electrode chamber has a substantially uniform width and a varying or non-uniform depth. Still other preferred embodiments employ an electrode chamber of non-uniform width and non-uniform depth. Other preferred embodiments include but are not limited to an electrode chamber defined by one or more non-linear walls, for example, a series of faces or facets, some or all having non-uniform dimensions; or wherein the electrode chamber has a curved cross-section, such as, for example, a half-circular cross-section, that varies axially, as, for example, a half-cone-shaped chamber.

In still other preferred embodiments, the electrophoretic devices comprise a separation chamber for receiving a fluid containing one or more analytes. The separation chamber has a non-uniform cross-section flow channel, that is to say, the cross-sectional area of the separation chamber varies axially along the channel. Two or more electrodes are separated from the chamber by a conductive material. The electrodes are operative to generate an electric field in the separation chamber when energized, wherein the non-uniformity of the separation chamber is operative to establish a gradient in an electric field. The non-uniformity of the separation chamber further leads to a gradient in the hydrodynamic force that exists as a result of flowing a fluid through the chamber. In certain preferred embodiments, an electrode chamber is separated from the separation chamber by a porous, conductive membrane, which in certain preferred embodiments is substantially planar. The electrodes are positioned proximate the electrode chamber and are operative to generate an electric field in the electrode channel. The electrodes generate an electric field in the electrode chamber which is communicated to the separation chamber, where the non-uniformity of the separation chamber induces a gradient in the electric field. The separation chamber in certain preferred embodiments has a substantially uniform height (height here meaning the direction normal to the plane of the membrane) and a non-uniform or non-constant width (width here meaning the direction perpendicular to the overall direction of flow and parallel to the plane of the membrane). In other preferred embodiments, the separation chamber has a substantially uniform width and a varying or non-uniform height. Still other preferred embodiments employ a separation chamber of non-uniform width and non-uniform height. Other preferred embodiments include a separation chamber defined by one or more non-linear walls, for example, a series of faces or facets, some or all having non-uniform dimensions; or wherein the separation chamber has a curved cross-section, such as, for example, a half-circular cross-section, that varies axially, as, for example, a half-cone-shaped chamber.

In certain preferred embodiments encompassing a non-uniform separation chamber, the electrode chamber has a uniform cross-section flow channel. In other such preferred embodiments, the electrode chamber is itself non-uniform, that is, the electrode chamber has a non-uniform cross-section flow channel. The non-uniformity of the electrode chamber will itself create a gradient in the electric field, the shape of which will in turn be affected by the non-uniformity of the separation chamber. The electrode chamber in certain preferred embodiments has a substantially uniform depth (depth here meaning the direction normal to the plane of the membrane) and non-uniform width. In other preferred embodiments, the electrode chamber has a substantially uniform width and a varying or non-uniform depth. Other preferred embodiments include an electrode chamber defined by one or more non-linear walls, for example, a series of faces or facets, some or all having non-uniform dimensions; or wherein the electrode chamber has a curved cross-section, such as, for example, a half-circular cross-section, that varies axially, as, for example, a half-cone-shaped chamber.

In accordance with yet another preferred embodiment, a device for focusing a charged analyte comprises a separation chamber and an electrode array isolated from the separation chamber and operative to establish an electric field gradient in the separation chamber. The separation chamber typically comprises an inlet for introducing a first fluid into the separation chamber and an outlet for exiting the first liquid from the separation chamber.

In certain preferred embodiments encompassing devices comprising an electrode array as just described, the electrophoretic devices further comprise an electrode chamber, with a permeable material between the separation chamber and the electrode chamber. The electrode array is positioned within or proximate the electrode channel, typically remote from the permeable membrane, and is operative to be energized to establish an electric field gradient in the first chamber through the permeable membrane. The electrode chamber typically has an inlet and an outlet for passing a fluid sample through the chamber, i.e., an inlet for introducing liquid into the electrode chamber and an outlet for exiting the liquid from the electrode chamber.

Still other preferred embodiments incorporate any combination of a configured separation chamber, a configured electrode chamber, and an electrode array, each in accordance with any of the embodiments described herein, for establishing the gradient in the electric field. Other suitable means for creating and establishing an electric field gradient in the separation chamber will be readily apparent to those of skill in the art, given the benefit of the present disclosure.

In accordance with a method aspect, electrophoretic devices as disclosed immediately above are employed, with molecular sieve in the separation chamber. A fluid sample is flowed through the separation chamber. An electric field gradient is established and maintained (with or without adjustment or change during the focusing process) in the focusing chamber, for example, by energizing the electrodes of the optional electrode array of the electrode chamber, whose voltages typically are individually monitored and controlled. Optionally, for example, the voltage applied to each electrode is controlled by a computer-controlled circuit board or suitable processor or the like in operative connection to a suitable voltage source. Certain preferred embodiments of such methods simultaneously focus multiple charged analytes from a fluid sample, including at least two analytes having the same or similar charge to mass ratio or electrophoretic mobility but different molecular weights. Each of the two analytes is focused in the chamber at a stable position spatially separated from the focusing location of the other. In accordance with certain preferred embodiments of such methods, a fluid sample containing multiple charged analytes of different molecular weights but having the same or similar charge to mass ratio or electrophoretic mobility is caused to flow through the separation chamber with a fixed or soluble molecular sieve, such that hydrodynamic force of the fluid flow is opposed by a gradient in the electric field and by the molecular sieve. It should be understood that reference above and elsewhere herein to a fluid sample passed through the separation or focusing chamber can mean a single fluid sample passed one or more times through the chamber or a series of two or more fluid samples passed in turn through the chamber.

In accordance with another method aspect, electrophoretic devices as disclosed above are employed with molecular sieve in the separation chamber, for focusing a charged analyte in the separation chamber at a stable position that, for a given set of focusing process parameters (e.g., sample fluid flow rate, composition and/or pH, electric field gradient strength and/or configuration, chamber configuration, etc.) is shifted from the location at which it would focus under the same set of process parameters absent the molecular sieve.

In accordance with certain preferred embodiments, devices and methods are provided, whereby two or more proteins or other biomacromolecules which have the same or similar charge to mass ratios or electrophoretic mobilities but different size, can be focused from the same fluid sample in the separation chamber of a device as disclosed above. Each such biomacromolecule is concentrated at a location in the chamber spatially separated from the locations at which others of the biomacromolecules are focused. In accordance with the principles disclosed above, the focusing locations of the different biomacromolecules are stable during the focusing process, that is, each of such analytes can be held at its respective focusing location in the chamber during and after the focusing process.

### BRIEF DESCRIPTION OF THE DRAWINGS

The foregoing aspects and many of the attendant advantages of the methods and devices disclosed here will be appreciated with reference to the following detailed description, when taken in conjunction with the accompanying drawings, wherein:

FIG. 1 is a graph illustrating the principles of EFGF;

FIG. 2 is an exploded view of an exemplary device;

FIG. 3 is a schematic of a system capable of functioning as a “notch filter;”

FIG. 4 illustrates the concept of the SPUR multiplexer;

FIG. 5 is a graph further illustrating the principles of EFGF;

FIG. 6 is a series of images showing three injected proteins coming into focus;

FIG. 7A-C are top views, a cross sectional side view, and a cross-sectional end view, respectively, of an exemplary device;

FIG. 8 is a schematic illustration of an exemplary system;

FIG. 9 is a schematic illustration of an exemplary dual-device system;

FIG. 10 is a series of images showing focused bands being eluted from an exemplary separation chamber;

FIGS. 11A and 11B are top and isometric views, respectively, of a diverter manifold of an exemplary system;

FIGS. 12A and 12B are top and isometric views of a general fluidic interface manifold of an exemplary system;

FIGS. 13A and 13B are top and isometric views of a top block of an exemplary system;

FIG. 14 is a separation channel layer of an exemplary system;

FIGS. 15A and 15B are top and isometric views of a bottom block of an exemplary system

FIG. 16 is an exploded isometric view of an exemplary system incorporating the elements illustrated in FIGS. 11-15;

FIG. 17 is a schematic of an exemplary system;

FIG. 18 is a schematic of an exemplary dual-device system;

FIG. 19 is a schematic of an alternative dual-device system;

FIG. 20 is a schematic perspective view of a first embodiment of the devices disclosed here;

FIG. 21 is a schematic perspective view of another embodiment of the devices disclosed here;

FIG. 22A is an exploded view of another embodiment of the devices disclosed here;

FIGS. 22B-22E are schematic perspective views of selected components of the device illustrated in FIG. 22A;

FIG. 23 is an elevation view, partly in section, of the device of FIGS. 22A-22E in assembly;

FIGS. 24A and 24B are front and back plan views, respectively, of the device of FIGS. 22A-22E and 23 in assembly;

FIGS. 25A and 25B are views, partially in section, of the device of FIGS. 22A-22E, 23 and 24A-24B, in assembly, taken through line 6A-6A in FIG. 23 and line 6B-6B in FIGS. 24A and 24B, respectively,

FIGS. 26A-26F present schematic representations and graphical representations of two approaches for conducting electric field gradient focusing in accordance with certain embodiments of the devices and methods disclosed here;

FIG. 27 is a schematic drawing of another embodiment of a device in accordance with the present disclosure;

FIGS. 28A and 28B each is a graphical representation of the field strength profile and potential profile, respectively, of a linear field gradient (15.5 v/cm²) in accordance with another embodiment of the methods and devices disclosed here;

FIG. 29 is a schematic representation of the resistance between two adjacent electrodes in another embodiment of the methods and devices disclosed here;

FIG. 30 is a schematic diagram of a representative electric field gradient focusing gradient control model of an embodiment of the methods and devices disclosed here;

FIG. 31 is a schematic diagram of a representative electric field gradient focusing gradient control circuits;

FIG. 32 is a circuit diagram of a representative controller unit;

FIG. 33 is a circuit diagram of a representative controller unit;

FIG. 34 is a schematic illustration of a representative DAC board circuit diagram illustrating connections,

FIGS. 35A and 35B are schematic illustrations of a representative DAC board circuit diagram illustrating components;

FIG. 36 is a schematic illustration of representative configurations for other preferred embodiments of the device;

FIG. 37 is a diagrammatic sectional view of a representative device.

### DETAILED DESCRIPTION OF CERTAIN PREFERRED EMBODIMENTS

Unless otherwise indicated or unless otherwise clear from the context in which it is described, aspects or features disclosed by way of example within one or more aspects or preferred embodiments should be understood to be disclosed generally for use with other aspects and embodiments of the devices and methods disclosed herein. Also, in accordance with traditional patent usage, the use of the indefinite article “a” in the following is intended to mean one or more than one, that is, at least one, unless otherwise clear from the context in which it is used. It should be understood that the mere usage of the phrase “at least one” or like phrases, in certain instances, is alone not an indication that usages of the individual article “a” in other instances means only one.

In operation under suitable focusing process parameters, the chamber or chambers of the devices disclosed above typically are filled with liquid sufficiently electrically conductive to establish an electric field gradient in the separation chamber, for example when the electrodes are energized. For embodiments encompassing an electrode chamber separated from the separation chamber by a permeable membrane, the permeable membrane preferably is operative to establish selective communication between the separation chamber and the electrode chamber, at least sufficiently to provide selective mass transport between the chambers, but prevents the target analyte from passing to the electrode chamber. The chambers typically are elongate and partly or wholly overlying one another in their longitudinal dimension. The optional electrode array typically includes a plurality of electrodes arranged along the chamber length, and each electrode optionally is capable of being individually controlled, i.e., energized at a level selected independently of the energization level of other electrodes of the array. In certain preferred embodiments the electrode array is operative to generate an electric field gradient profile which can be dynamically controlled.

Preferred embodiments of the devices disclosed here are suitable for focusing charged analytes and for separating mixtures of charged analytes such as DNA or other naturally occurring or artificial biomacromolecules having the same or similar charge to mass ratios and electrophoretic mobilities, i.e., the same or similar magnitude of charge per unit length of the biomacromolecules (or charge per unit of molecular weight) at a given pH of the buffer solution in which the sample is presented. Without wishing to be bound by theory of operation, it currently is understood that the molecular sieve in the separation chamber during the focusing process, acting cooperatively with other forces operative on the analytes, i.e., under the focusing process parameters in use, including at least the hydrodynamic force of the fluid sample flow in the chambers, pH, the electric field strength and configuration, the configuration of the chambers, etc, causes different analytes to focus at spaced locations in the separation chamber. Thus, employing such molecular sieve in the separation chamber with suitable focusing process parameters, analytes that otherwise would focus at the same (i.e., overlapping or insufficiently spaced) locations instead focus at different locations. Analytes that would otherwise be unseparated by traditional electric field gradient focusing (EFGF) devices and methods can be focused at stable, separate locations according to their molecular weights by the devices and methods disclosed here. Stable means that the location remains substantially constant as long as the conditions of the analysis remain substantially constant The devices disclosed here may also be used to separate and focus charged analytes wherein some but not all of the charged analytes share a similar mobility. It will be understood that, as used herein, the term “focusing process parameters” is intended to mean all parameters and characteristics of an EFGF analysis that influence the focusing of a charged analyte in a system not incorporating or using molecular sieve, unless otherwise indicated or made apparent by the context in which it is used.

“Electrophoretic device” as used herein refers to any device which employs opposing hydrodynamic and electrophoretic forces to affect the location of a charged analyte within the device. As used here, the term “focus” and other forms of that word are used generally to mean concentrating a desired analyte (i.e., a target species dissolved or suspended in a sample fluid) in the separation chamber of an electrophoresis device in accordance with the above disclosure. It will be readily understood that this inherently includes separating that analyte from the carrier fluid and typically from one or more other analytes that do not concentrate at the same location in the chamber under the focusing process parameters employed. The term “separating” and other forms of that word, unless otherwise indicated by context or the like, generally are used to describe the result of the present invention employing molecular sieve in the separation chamber, i.e., separating the desired analyte from the sample fluid and, in certain preferred embodiments, from other analytes having the same or similar electrophoretic mobility but different molecular weight. As used here, two analytes are said to concentrate or focus at the same location in the separation chamber under the processing parameters employed, when they focus at locations wholly or partially overlapping one another or so close as to unduly inhibit isolating one species from the other such as by drawing a first one of the analytes out of the chamber via a main flow exit port or via a selectively opened side port or the like.

In certain preferred embodiments, an electrophoretic device includes a focusing chamber having an electrode array. The focusing chamber is a divided chamber that includes a separation chamber and an electrode chamber separated by a permeable material. Charged analyte separation and focusing occurs in the separation chamber, which includes the molecular sieve. The electrode chamber includes electrodes, optionally an array of electrodes, for generating a focusing electric field gradient. The separation chamber is in electrical communication and mass or ionic communication with the electrode chamber through the permeable material. “Electrical communication” as used herein refers to the ability of the electric field gradient that is generated by the electrode array to be transferred, or to have an effect, within the separation chamber, and may be by any means which accomplishes this. The permeable material retains analytes in the separation chamber and is permeable to certain analytes such that the electrode chamber and separation chambers are in communication as noted above. Generally, a fluid, e.g., an eluant, is introduced into and flows through the separation chamber containing the charged analyte. The eluant flow is opposed to the direction of electrophoretic migration of the analyte.

A given set of focusing process parameters, as noted above, includes all parameters, both dynamic and non-dynamic, that affect the location of a focused band of charged analyte in the separation chamber, other than the influence of the molecular sieve. With the influence of the molecular sieve, the focusing location is different than it would be in the absence of the molecular sieve. All such parameters are encompassed by the term “focusing process parameters” unless otherwise noted or otherwise clear from context. Such factors include, for example, dynamic factors, or factors that are capable of being changed, such as the particular characteristics such as the shape and strength of the electric field gradient; the composition, concentration and pH of the first liquid; the flow rate of the first liquid; the composition, concentration and pH of the second liquid, the flow rate of the second liquid; and other such dynamic factors. The parameters that make up the focusing process parameters further include nondynamic factors such as the dimensions of the separation chamber and optional electrode chamber; and other such nondynamic factors.

In simultaneous focusing in a separation chamber of multiple charged analytes having the same or similar charge to mass ratios, the composition and amount of molecular sieve is chosen such that the location of the stationary focused band of each such analyte is shifted in the chamber to a different degree. It should be understood, however, that reference here to each of multiple analytes being shifted to a different degree does not exclude the possibility that in any given stationary focused band there may be more than one analyte, that is, there may be analyte mixtures for which the devices and methods disclosed here are operative to establish focuses bands of subsets of the analytes, each subset containing one or more of the analytes. Typically the analytes are separated on the basis of their molecular weights or masses. This is particularly useful for separating analytes that have the same or similar mobilities that would not adequately separate in a traditional EFGF device absent the sieve.

Molecular sieves include any medium or substance, for example suitable organic or inorganic polymer or the like, by which such shifting of the focusing location is achieved. The molecular sieve is selected for its ability to, shift the location of the stationary focused band of analyte for simultaneous focusing of multiple charged analytes. Preferably, a molecular sieve is chosen such that the amount to which the stationary focused bands of analyte are shifted for a given set of focusing conditions varies with the size or molecular weight of the analyte. Preferably the degree of shift varies proportionally with the molecular weight of the analyte, for example, such that each stationary focused band of charged analyte is focused at a stable location separate from the other charged analytes. Factors that affect the selection of a particular molecular sieve at a particular concentration include, for example, the size of the molecules to be separated and focused, the pH at which the system is operated, and other such relevant factors that will be apparent to those skilled in the art, given the benefit of this disclosure. In certain preferred embodiments, the molecular sieve comprises a gel, which may be either an organic gel or an inorganic gel or a combination of organic and inorganic gel. The gel may be a fixed gel. A fixed gel optionally may be polymerized within the first chamber, such that it does not substantially flow or move when fluid sample is flowed through the first chamber. Alternatively, the gel may be a soluble gel that is dissolved in the first liquid, such that the gel flows with the first liquid when the first liquid flows through the first chamber. In certain embodiments, the soluble gel is introduced into the chamber and resides there during focusing. As used herein, the term “soluble gel” refers to a gel that is soluble or dissolved in a liquid or fluid, and further refers to gels that form suspensions, emulsions, colloids, and the like. Typically, soluble gels comprise polymers having little or no crosslinking. In certain preferred embodiments, the gel will be comprised of molecules having a molecular weight of between about 2000 and about 100,000. Suitable gels include, for example, linear polyacrylamide, polyvinyl alcohol, methyl cellulose and other derivatized celluloses, and the like. Other suitable molecular sieves include microporous structures composed of either crystalline aluminosilicate, chemically similar to clays and feldspars and belonging to a class of materials known as zeolites, or crystalline aluminophosphates derived from mixtures containing an organic amine or quaternary ammonium salt, or crystalline silicoaluminophosphates which are made by hydrothermal crystallization from a reaction mixture comprising reactive sources of silica, alumina and phosphate, and the like. Those of ordinary skill in the art will be able to select suitable gels and sieves through routine experimentation, utilizing known methods, for example by the methods described in Ackers et al., “Determination of stoichiometry and equilibrium constants for reversibly associating systems by molecular sieve chromatography,” Proc. Nat. Acad. Sci. USA 53: 342-349 (1965), the entire disclosure of which is hereby incorporated by reference for all purposes. Other suitable sieves will be readily apparent to those of ordinary skill in the art, given the benefit of the present disclosure.

Without wishing to be bound by theory, it is presently understood that the device is based on the principle of opposing two counteracting forces to create a dynamic equilibrium point. The force in one direction is a resultant of bulk fluid flow, commonly referred to as chromatographic flow, which imposes a hydrodynamic velocity on solutes in the stream. The magnitude of the hydrodynamic velocity is proportional to the hydrodynamic radius or apparent size of the solute, and is adjustable with changes in the rate of chromatographic flow. In the opposite direction, an electrophoretic velocity is induced with the application of a voltage to the stream containing solutes. The electrophoretic velocity is proportional to the molecular charge of the solute, which is adjustable with changes in solvent pH or composition. At a point in the separation path where the opposing velocities are equal in magnitude, yielding a net zero velocity, is the focal point for a particular solute. The focal point is one of a dynamic equilibrium for the solute, whereby any movement from that point results in a non-zero velocity and a restoring force.

Without wishing to be bound by theory, the molecular sieve in preferred embodiments can be said to apply a focus position-shifting force on the analyte along the direction of fluid sample flow, where the magnitude of such force for a particular molecular sieve material is generally proportional to the size or molecular weight of a target analyte being focused (or held) in the separation chamber and where the magnitude of such force is not (or not as) related to the charge-to-mass (or charge per unit of molecular weight) of the analyte. Charged analytes in a fluid sample can in this way be retained and focused in the separation chamber at locations spatially separated from each other sufficiently to permit each to be readily drawn off or removed from the chamber with little or none of the other focused analytes. Typically, the charged analytes are separated by the molecular sieve, in conjunction with the other focusing process parameters and conditions, and focused at positions along the length of an elongate separation chamber in the general order of their apparent molecular weights. It will be within the ability of those skilled in the art, given the benefit of this disclosure, in some cases with routine trial and error or similar selection aids, to select materials suitably operative as molecular sieves for the intended target analyte(s) under a given set of process parameters. Accordingly, in certain preferred embodiments of the methods and devices disclosed here, each of multiple analytes having the same or similar charge to mass ratios or electrophoretic mobilities and different molecular weights, can be simultaneously focused from a fluid sample at different, separate locations along the length of an elongate separation chamber. Each such analyte can be held indefinitely at its respective focusing location against the flow of the fluid sample. In accordance with certain preferred embodiments, spaced focusing positions of such target analytes can be moved in the chamber to different, stable, separate locations, by suitable control of one or more of the operative focusing forces, e.g., by adjusting the electric field gradient strength or configuration, or the hydrodynamic force of the sample fluid flow, such as by changing its flow rate, or by changing the characteristics of the molecular sieve.

In accordance with certain preferred embodiments, electrophoresis devices comprising molecular sieve in the separation chamber are provided that are operative to perform electric field gradient focusing (EFGF), employing a counter-balance of chromatographic flow against electromigration to create high resolution, free-solution separation and focusing functionality for a broad range of analytes in buffer systems, including simple buffer systems. Such devices comprise a separation chamber as a focusing chamber and a nom-uniform electrode chamber separated from the separation chamber by a porous or conductive membrane, e.g., a suitably functionalized dialysis membrane, Nafion® or other ion exchange membrane, which in certain preferred embodiments is substantially planar in configuration. Electrodes are positioned proximate the electrode chamber, i.e., in or near the non-uniform electrode chamber such that the electrode chamber is operative to establish an electric field gradient, which is communicated to the separation chamber. The membrane is effective to pass electrical current and electrolyte ions (e.g., tris-phosphate buffer ions), but not the analyte, i.e., not the target molecule of interest being focused or concentrated in the separation chamber. The separation chamber comprises molecular sieve operative to shift the location at which a particular charged analyte focuses. Certain preferred embodiments of the electrophoresis devices disclosed here are operative to capture and concentrate a sample, as well as route (i.e., release) the sample from the chamber, and have applicability to processes in biotechnology, pharmaceutical or other scientific research and development areas as well as industrial production and testing applications. Certain preferred embodiments of the electrophoresis devices disclosed here provide a dynamic platform for preconcentration and routing of target solutes for subsequent analysis, and can serve as a sample preparation tool. Certain preferred embodiments of the electrophoresis devices disclosed here are substantially planar in configuration, the conductive, porous membrane being substantially flat with the sample flow channel above and the electrode chamber below.

As described herein, charged analytes can be separated and focused by electrophoretic devices incorporating molecular sieve in the separation chamber. Suitable devices and methods include those described, for example by the devices and methods found in U.S. Pat. Nos. 5,298,143 and 6,277,258, in U.S. Application Ser. No. 60/440,150, entitled “Devices and Methods for Focusing Analytes in an Electric Field Gradient,” filed on 15 Jan. 2003; U.S. Application Ser. No. 60/440,105, entitled “Method and Apparatus for Determining the Isoelectric Point of a Charged Analyte,” filed on 15 Jan. 2003; U.S. Application Ser. No. 60/430,493, entitled “Electrophoresis Device, System and Method for Sample Management and Hyphenation of Analytical Instruments,” filed on Dec. 2, 2002; U.S. Application Ser. No. 60/447,997, (not yet assigned), entitled “Electrophoresis Device, System and Method for Sample Management and Hyphenation of Analytical Instruments,” filed on Feb. 18, 2003; U.S. Application Ser. No. 60/471,616, entitled “Electrophoresis Device, System and Method for Sample Management and Hyphenation of Analytical Instruments,” filed on May 19, 2003; U.S. Application Ser. No. 60/471,681, entitled “Method and Apparatus for Determining the Isoelectric Point of a Charged Analyte,” filed on May 19, 2003; U.S. Application Ser. No. 60/471,597, entitled “Devices and Methods for Focusing Analytes in an Electric Field Gradient,” filed on May 19, 2003; or in U.S. Application Ser. No. 60/471,595, entitled “Electrophoresis Devices and Methods for Focusing Charged Analytes,” filed on May 19, 2003, all of which are incorporated herein in their entirety for any and all purposes.

Certain preferred embodiments comprise a separation chamber separated from a non-uniform electrode chamber by a substantially planar porous, conductive membrane. The separation chamber comprises molecular sieve. The electrode chamber is non-uniform axially, that is to say, the cross-section of the separation chamber varies along the axial length of the channel, such that a gradient is established in an electric field that is generated by the electric field in the electrode chamber and communicated into the separation channel. The electrode channel in certain preferred embodiments has a substantially uniform depth (depth here meaning the direction normal to the plane of the membrane) and a non-uniform or non-constant width (width here meaning the direction perpendicular to the overall direction of flow and parallel to the plane of the membrane). In other preferred embodiments, the electrode chamber has a substantially uniform width and a non-uniform depth. In yet other preferred embodiments, the width and the depth are both non-linear, and may include side walls and a bottom wall that are each nonlinear in the same fashion or to differing degrees, multiple facets that are each non-linear to the same or different degrees, or may form a cone-like shape wherein the walls are curved in a direction normal to the axial direction and non-linear in the axial direction. Combinations of these are also possible. As discussed further below, it will be within the ability of those skilled in this technology area, given the benefit of this disclosure, to employ suitable separation channel geometry, sample flow rate, sample loading, as well as field strength in the electrode chamber to achieve good separation resolution in a short processing or “focusing” time.

In other preferred embodiments, the porous, conductive membrane need not be planar. The electrode chamber in these embodiments is non-uniform in width and substantially uniform in depth. The side walls, i.e., those adjacent the membrane, in certain preferred embodiments may be linear and nonparallel, or in shape. Other suitable configurations for the electrode chamber will be readily apparent to those of ordinary skill in the art, given the benefit of the present disclosure.

The separation chamber is typically a uniform cross-section flow channel or chamber, but may in certain preferred embodiments be non-uniform and can comprise any of the configurations described above for the electrode chambers.

In still other preferred embodiments, the device comprises a separation chamber separated from a non-uniform electrode chamber by a porous, conductive membrane. The separation chamber typically is located alongside or adjacent the electrode chamber. In certain preferred embodiments, the separation chamber may be partially or completely located within the electrode chamber, as, for example, a separation chamber enclosed partially or completely by the membrane and located within the electrode chamber. For example, the electrode chamber may be substantially conical, with the separation chamber located entirely within the electrode chamber and separated from the electrode chamber by a tubular membrane, such as, for example, dialysis membrane tubing. In other preferred embodiments, the electrode chamber comprises an electrode array, typically positioned proximate or within the electrode chamber, operative to generate an electric field gradient in the electrode chamber which is then further affected by the non-uniformity of the electrode chamber. The electrode chamber and the separation chamber may be of any of the configurations described above.

In accordance another preferred embodiment, electrophoresis devices are provided that comprise a non-uniform cross-section separation chamber as a focusing chamber and an electrode chamber separated from the separation chamber by a porous or conductive membrane as described above, with electrodes proximate the electrode chamber operative to establish an electric field which is communicated to the separation chamber. The non-uniformity of the separation chamber establishes a gradient in the electric field along the separation chamber, as well as creating a gradient in the hydrodynamic force brought about by the flow of fluid through the separation chamber. The membrane is effective to pass electrical current and electrolyte ions (e.g., tris-phosphate buffer ions), but not the analyte, i.e., not the target molecule of interest being focused or concentrated in the separation chamber. Certain preferred embodiments of the electrophoresis devices disclosed here are operative to capture and concentrate a sample, as well as route (i.e., release) the sample from the chamber, and have applicability to processes in biotechnology, pharmaceutical or other scientific research and development areas as well as industrial production and testing applications. Certain preferred embodiments of the electrophoresis devices disclosed here provide a dynamic platform for preconcentration and routing of target solutes for subsequent analysis, and can serve as a sample preparation tool. Certain preferred embodiments of the electrophoresis devices disclosed here are substantially planar in configuration, the conductive, porous membrane being substantially flat with the separation flow channel above and the electrode chamber below.

The separation chamber is non-uniform axially, that is to say, the cross-section of the separation chamber varies along the axial length of the channel, such that a gradient is established in an electric field that is generated in the separation channel by the electric field in the electrode chamber. The separation channel in certain preferred embodiments has a substantially uniform height (height here meaning the direction normal to the plane of the membrane) and a non-uniform or non-constant width (width here meaning the direction perpendicular to the overall direction of flow and parallel to the plane of the membrane). In other preferred embodiments, the separation channel has a substantially uniform width and a non-uniform height. In yet other preferred embodiments, the width and the height are both non-linear, and may include side walls and a top wall that are each nonlinear in the same fashion or to differing degrees, multiple facets that are each non-linear to the same or different degrees, or may form a cone-like shape wherein the walls are curved in a direction normal to the axial direction and non-linear in the axial direction. Combinations of these are also possible. As discussed further below, it will be within the ability of those skilled in this technology area, given the benefit of this disclosure, to employ suitable separation channel geometry, sample flow rate, sample loading, as well as field strength in the electrode chamber to achieve good separation resolution in a short processing or “focusing” time.

The electrode chamber in this aspect is preferably a uniform cross-section flow channel or chamber. In other preferred embodiments, the electrode chamber non-uniform and can comprise any of the embodiments described above for the separation chamber.

In certain preferred embodiments, the focused analytes can be eluted from the electrophoretic focusing device through one or more additional ports positioned midway along the separation chamber, typically between the inlet port and the outlet port. Basically, the desired analyte can be focused to a region of the chamber from which the analyte can be eluted through a port. Analytes can be eluted from the separation chamber by electric field, pressure, vacuum, or other motive force.

In preferred embodiments incorporating a non-uniform separation chamber, the magnitude of the hydrodynamic velocity is adjustable with changes in the rate of chromatographic flow and/or with changes in the shape and/or size of the separation chamber. The hydrodynamic velocity will vary throughout the separation chamber as a result of the non-uniformity of the separation chamber. The hydrodynamic radius of an analyte is independent of the charge, and thus is independent of the electrophoretic velocity of the analyte. Thus, the provision of a gradient in the electric field and in the flow rate advantageously provides two independent means of achieving separation of charged analytes, thus increasing the likelihood of being able to separate multiple analytes. In establishing a desired electric field gradient, those skilled in the art will recognize that some compensation must be made in the gradient-establishing parameters (i.e. the shape of the electrode chamber or the settings for the electrode array) to address the perturbation or influence in the electric field caused by the non-uniformity of the separation chamber where such is configured. That is to say, once a particular separation chamber configuration is desired, such that a desired hydrodynamic force gradient is established, that configuration must be taken into account when determining the appropriate configuration of the electrode chamber to achieve the desired shape of the electric field gradient. For example, while a hyperbolic electrode chamber would, in conjunction with a uniform or non-configured separation chamber, lead to a linear field gradient, the electrode chamber must deviate from hyperbolic to achieve a linear field gradient in the presence of a non-uniform separation chamber. Determination of suitable chamber configurations will be readily apparent to those of skill in the art, given the benefit of the present disclosure.

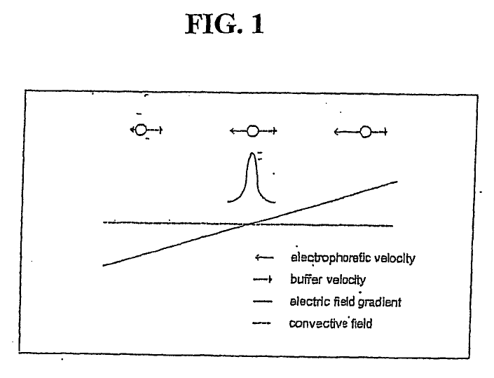

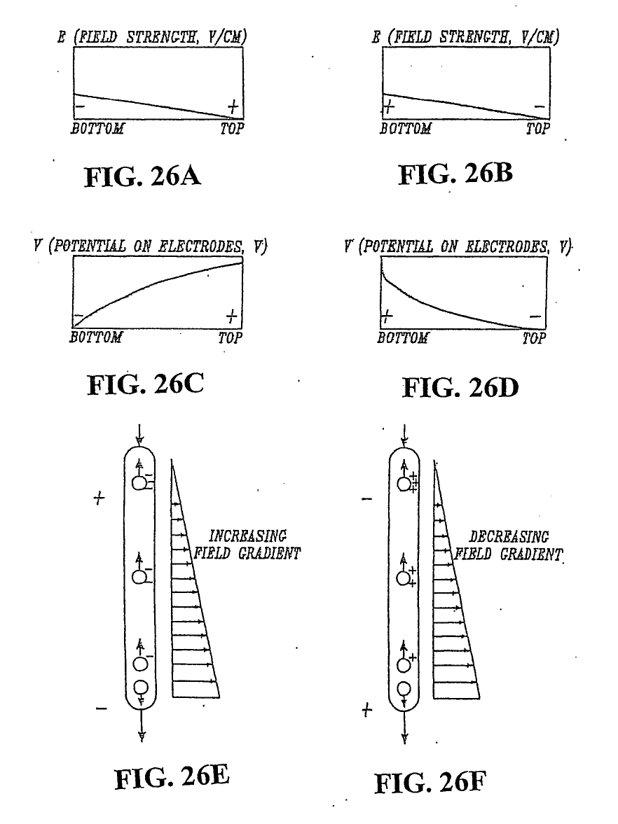

When the collected and held-in-place solute or analyte is to be released, the electrical field can be decreased or eliminated or the flow rate increased. The concept of electric field gradient focusing is illustrated in FIG. 1, where a constant bulk fluid flow is counteracted by a linear gradient in the electric field strength. A bulk buffer flow pushes solute to the right, while being counteracted by an electrophoretic force in the opposite direction. The magnitude of the electrophoretic force varies along the axis of the separation chamber. FIG. 26 further illustrates this concept. First, negatively charged proteins focus in an increasing field gradient with the electric field in the same direction as the convective flow of buffer (A, C, E). Second, positively charged proteins focus in a decreasing field gradient with the electric field in opposite direction as the convective flow (B, D, F). The amount of charge carried on protein molecules are closely related to the pH of the buffer and are generally different from species to species. The migration rate is directly proportional to the amount of charge carried which is generally different from specie to specie. Therefore, distinct stationary accumulation zones for differently charged species are generated along the column. In order to focus the charged protein in the chamber, the direction of electric field, the slope of field gradient and the pH of the elution buffer must be matched. Otherwise, the target protein will be flushed out or concentrated at the very top of the column, allowing no separation at all.

In certain preferred embodiments, a pair of electrodes or optionally an array of electrodes is utilized to generate the electric field, with a gradient arising by means of the configuration of the separation chamber, optionally in conjunction with a configured electrode chamber and/or electrode array, as described above. In such embodiments, the configuration of the separation chamber and/or the electrode chamber is itself subject to dynamic control, either by the user or by computer control. Such embodiments employ, for example, movable or pivotable walls such that the shape and size of the chamber can be altered during the course of a focusing run to provide dynamic control over the strength and/or shape of the electric field gradient. Where the separation chamber configuration is dynamic, the gradient in the hydrodynamic force is advantageously subject to dynamic control; providing still more flexibility to the separation methods available. Suitable configurations employing dynamically-controlled chamber configurations will be readily apparent to one skilled in the art, given the benefit of the present disclosure.

As discussed above, the electrodes in certain preferred embodiments are separated from the separation chamber by a membrane. Suitable membranes allow an electric field to be generated through the membrane material in the separation chamber while desired analytes, for example, macromolecules such as biomacromolecules, are retained in the separation chamber, that is, are not able to directly contact the electrodes. In certain preferred embodiments, the membrane is conductive to heat but not to bulk fluid flow. The membrane advantageously serves to isolate the electrodes from the separation chamber and optionally to avoid disruption of the laminar flow by gas generation or denaturation of charged analyte by contact with the electrodes. Suitable conductive materials include Nafion, cellulose based membranes, membranes having a MWCO of about 100-1000, etc. In certain preferred embodiments, the separation and electrode chambers are separated by the membrane. In such embodiments, the membrane is typically a permeable material. As used herein, a permeable material is one that allows communication through the permeable material while (1) desired analytes, for example macromolecules such as biomacromolecules, are retained in the separation chamber; (2) undesired contaminants can be dialyzed out of the separation chamber; and (3) desired molecules, for example buffer ions, etc., can be dialyzed into the separation chamber. In certain preferred embodiments, the permeable material is conductive to heat and buffer ions but not to bulk fluid flow. The permeable material advantageously serves to isolate the electrodes from the separation chamber to avoid disruption of the laminar flow by gas generation or denaturation of charged analyte by contact with the electrodes. Suitable permeable materials include permeable membranes such as dialysis membranes and ion exchange membranes. Other suitable permeable materials will be readily apparent to those of ordinary skill in the art, given the benefit of the present disclosure.

In operation, the device includes the flow of a first or sample fluid, typically a liquid, through the first or separation chamber, and the flow of a second or electrode fluid, also typically a liquid, through the second, or electrode chamber. Generally, the first liquid is an electrophoretic eluant (e.g., buffer solution) and the second liquid is a coolant. Suitable liquids include simple liquids such as buffered water, and complex fluids, for example mixtures of water and solvent, etc. The first liquid can be the same as or different from the second liquid. During focusing and separation, and depending on the requirements of the particular separation, the composition of either the first and/or the second liquid can be changed to achieve the desired result. Liquid flow through the separation chamber preferably opposes the direction of electrophoretic migration of the analyte and can be driven by any one of a variety of forces including electric field, pressure, vacuum, or other motive force. In a preferred embodiment, the direction of liquid flow through the separation chamber is opposite that through the electrode chamber.

In accordance with certain preferred embodiments, a fluid gradient can be used to provide increased separation between different bands of analytes. As used here, fluid gradient refers to variation in the composition of the fluid flowing through the separation chamber during the separation of the analytes. For example, in a separation using two solvents, A and B, the separation may begin with 100% solvent A. As the separation progresses, the amount of solvent B can be increased, e.g., linearly, step-wise, logarithmically, etc., such that the solvent composition introduced into the chamber includes both A and B. Typically, the amount of each solvent in the solvent gradient is controlled by varying the amount of solvent introduced into the chamber. The solvents typically are introduced into the chamber through one or more pumps or other suitable devices. In certain embodiments, it may be necessary to provide a mixing chamber so that the solvent can be mixed prior to introduction of the solvents into the devices described here. In certain embodiments, the solvent gradients are computer controlled to provide high precision for the separations. One skilled in the art, given the benefit of this disclosure, will be able to select suitable solvent gradients for use in the devices and methods disclosed here.

In certain preferred embodiments, a hydrodynamic force is applied to the first fluid by pumping the first fluid through the separation chamber. The first fluid typically is a liquid with flow rates ranging, e.g., from 0.1 to 10 μL/min. for analytical applications, and, e.g., from 10 to 200 μL/min. for preparative applications. The flow rate is chosen to provide the desired separation, in other words so that the hydrodynamic force, when combined with the effect of the molecular sieve in embodiments comprising such, counters the electric field gradient at a position between the weakest and the strongest part of the electric field. In this fashion, the analyte will be retained within the separation chamber. Factors that affect the choice of flow rate include, for example, the viscosity and density of the fluid, strength of the electric field gradient, net charge of the analyte, etc. Suitable flow rates can be readily determined by routine trial and error.

In accordance with certain preferred embodiments, solvents that are used in the devices and methods disclosed here may be degassed prior to separation of analytes. Without wishing to be bound by any particular scientific theory, it is believed that dissolved gases in the solvents can affect the reproducibility of the flow rates of the solvents. Thus, to achieve constant and reproducible flow rates, it may be necessary to remove at least some of the dissolved gases from any solvents prior to introduction of the solvents into the devices described here. The person of ordinary skill in the art, given the benefit of this disclosure, will be able to select suitable techniques for degassing the solvents including, but not limited to, vacuum filtration of the solvents, e.g., filtration through a fritted funnel, bubbling of inert gases, such as, for example, argon and nitrogen, through the solvents, and the like.

In accordance with certain preferred embodiments, a solvent gradient may be used to provide increased separation between different bands of analytes. As used here, solvent gradient refers to variation in the composition of the solvent during the separation of the analytes. For example, in a separation using two solvents, A and B, the separation may begin with 100% solvent A. As the separation progresses, the amount of solvent B can be increased, e.g., linearly, step-wise, logarithmically, etc., such that the solvent composition introduced into the chamber includes a mixture of both solvents A and B. Typically, the amount of each solvent in the solvent gradient is controlled by varying the amount of solvent introduced into the chamber. The solvents typically are introduced into the chamber through one or more pumps or other suitable devices. In certain embodiments, it may be necessary to provide a mixing chamber so that the solvents can be mixed prior to introduction of the solvents into the devices described here. In certain embodiments, the solvent gradients are computer controlled to provide high precision for the separations. One skilled in the art, given the benefit of this disclosure, will be able to select suitable solvent gradients for use in the devices and methods disclosed here.

In accordance with certain preferred embodiments, lipids may be introduced either in the solvent or in the loaded sample. Without wishing to be bound by any particular scientific theory, lipids typically are either hydrophobic, having only nonpolar groups, or can be amphipathic, having both polar and nonpolar groups. In embodiments where one or more analytes are uncharged, it may be necessary to introduce an amphipathic lipid into the sample. Again without wishing to be bound by any particular scientific theory, the nonpolar group of the lipid can associate with one or more uncharged analytes, e.g., through hydrophobic interactions, hydrogen bonding, dipolar interactions, and the like, while the polar group of the lipid typically remains free to provide an overall charge to the lipid-analyte complex. In certain embodiments, lipids are selected from phosphatidic acid, phospholipids and glycerophospholipids such as, for example, phosphatidylcholine, phosphatidylethanolamine, phosphatidylserine, cardiolipin, phosphatidylglycerol, phosphatidylinositol, and the like. In other embodiments, the lipids may include ether glycerophospholipids, cerebrosides, sphingolipids, and the like. One skilled in the art, given the benefit of this disclosure, will be able to select these and other suitable lipids for use in the devices and methods disclosed here.

In accordance with certain embodiments, the lipids can form micelles that may associate with one or more analytes. Without wishing to be bound by any particular scientific theory, because many lipids include a nonpolar group and a polar group, e.g., amphipathic lipids, when the lipids are placed into an aqueous environment, the lipids typically spontaneously associate with each such that the polar groups are positioned outward towards aqueous solvent and the nonpolar groups are positioned inward away from aqueous solvent. Typically, it is necessary to provide the lipids in a sufficient amount, e.g., a critical micelle concentration (CMC), such that micelles can spontaneously form. That is, when the lipids are present at concentration below the CMC, the predominate form is individual free lipids. When the lipids are present at a concentration greater than or equal to the CMC, the predominant form is micelles. Suitable CMC concentrations will be readily selected by those skilled in the art, given the benefit of this disclosure, and the CMC concentration typically depends on the type of lipid selected.

In accordance with certain preferred embodiments, the lipids may form vesicles, e.g., unilamellar (large unilamellar vesicles (LUVs), small unilamellar vesicles (SUVs)) or multilamellar vesicles. Such vesicles are typically characterized as including one or more bilayers formed when the nonpolar groups of the lipids associate with each other. Suitable methods for preparing vesicles will be readily selected by those skilled in the art, given the benefit of this disclosure, and include but are not limited to extrusion, sonication/extrusion, and the like.

In accordance with other preferred embodiments, in the presence of lipids, micelles and/or vesicles, the analytes can partition between the bulk solvent and the lipids, micelles and/or vesicles. For example, one or more portions of the analyte molecule can interact with a portion of the lipid to form an analyte-lipid complex. Typically an equilibrium is established between free analyte and analyte complexed with lipid. It may be possible to favor this equilibrium depending on the nature of the analyte and the nature of the lipid selected. For example, it is possible to favor the lipid-analyte complex by adding lipid in amounts far in excess of the analyte concentration to shift the equilibrium to form additional analyte-lipid complex. When the predominant form in solution is analyte-lipid complex, the position at which the analyte is focused typically will differ from the position at which free analyte will focus. In certain embodiments, lipid-analyte complex will focus at a position substantially less than free analyte, i.e., under similar separation conditions, free analyte typically can migrate further than analyte-lipid complex. One skilled in the art given the benefit of this disclosure will be able to select suitable lipids and suitable amounts of the lipids to favor, or disfavor, lipid-analyte complexes.

In accordance with certain preferred embodiments, lipids, micelles and/or vesicles can be added to a sample to separate analytes of similar molecular weights and/or similar overall charges. Without wishing to by bound by any particular scientific theory, in many instances analytes having similar molecular weights and/or similar overall charges will be difficult to separate from each other and typically will appear as a single band. To facilitate separation of such analytes, lipids, micelles and vesicles can be used. Because there is likely to be some differences between the analytes, e.g., differences in hydrophobicity, composition, three-dimensional structure, surface area properties, and the like, the analytes should interact differently with the lipids, micelles and/or vesicles. For example, if one of the analytes includes a large number of hydrophobic groups, such as amino acids leucine, alanine, valine, etc., then it is possible that these hydrophobic groups will interact more frequently with hydrophobic lipids to reduce entropically disfavored interactions with polar bulk solvent. Accordingly, the use of lipids, micelles and/or vesicles can provide for the ability to baseline separate two or more analytes that behave similarly in the devices provided here.

In accordance with other preferred embodiments, the lipids, micelles and/or vesicles can be used to focus an analyte in a different position than in the absence of any lipids, vesicles or micelles. This result may be desirable for low molecular weight analytes or highly charged analytes, for example, which are difficult to focus at or near a sampling port. For example, without wishing to be bound by any particular scientific theory, it may be difficult to prevent certain analytes from migrating out of the device due to small size, high charge, etc. In the presence of lipids, micelles and vesicles, the analyte-lipid complex can increase the effective size of the analyte, which can reduce its rate of migration in the devices disclosed here. After removal of the analyte-lipid complex, e.g., through an exit port or a sampling port, the analyte-lipid complex can be dissociated and the analyte can be isolated using methods routinely used by the person of ordinary skill in the art, e.g., centrifugation, dialysis, etc. It will be within the ability of the person of ordinary skill in the art, given the benefit of this disclosure, to select and use suitable lipids, micelles and vesicles, and suitable amounts of these compounds, to control migration of one or more analytes in the devices disclosed here.

In accordance with yet other preferred embodiments, the lipids, micelles and/or vesicles can be used to separate two or more analytes having very similar migration behavior, e.g. two or more analytes that focus at the same position within the chamber. This result may be desirable for samples comprising two or more analytes that are similarly charged, for example, and difficult to separate from each other. For example, without wishing to be bound by any particular scientific theory, it may be difficult to separate analytes having similar charges even if those analytes have different physical or physicochemical properties, e.g., different hydrophobicities, secondary or tertiary structures, etc. In the presence of lipids, micelles and vesicles, the analyte-lipid complex can increase the effective size of the analyte, which, in certain embodiments, can reduce its rate of migration in the devices disclosed here. Because different analytes may interact differently with the lipids, due to the differences in the physical properties of the analytes, for example, it may be possible to favor the lipid-analyte complex for one analyte and favor free analyte for another analyte so that the two analytes may be separated from each other. After removal of the analyte-lipid complex, e.g., through an exit port or a sampling port, the analyte-lipid complex can be dissociated and the analyte can be isolated using methods routinely used by the person of ordinary skill in the art, e.g., centrifugation, dialysis, etc. It will be within the ability of the person of ordinary skill in the art, given the benefit of this disclosure, to select and use suitable lipids, micelles and vesicles, and suitable amounts of these compounds, to control migration of one or more analytes in the devices disclosed here.

As noted above, the device may include an optional electrode array. As mentioned earlier, unless otherwise indicated or unless otherwise clear from the context in which it is described, aspects or features disclosed by way of example within one or more aspects or preferred embodiments should be understood to be disclosed generally for use with other aspects and embodiments of the devices and methods disclosed herein. As used herein, the term “electrode array” refers to a plurality of electrodes, that is to say, more than two electrodes, arranged so as to generate an electric field gradient in the separation chamber. The electric field generated by the electrode array can be DC, AC, or otherwise modulated in time including asymmetric (out of phase) field modulation. The specific nature of the electrode (i.e., size and shape) is not critical. Suitable electrodes include pin-shaped and staple-shaped electrodes, among others. In one embodiment, the electrode array includes a linear array of electrodes (e.g., 50 electrodes arranged linearly) along an axis parallel to the direction of analyte migration. In addition to arrays having electrodes arranged in line with even spacings from one to the next, suitable arrays also include arrays in which the electrodes are not in line and which are not separated by even spacings. Other configurations of electrodes, including two-dimensional electrode arrays, are also within the scope of the devices and methods. Two-dimensional arrays include arrays having rows and columns of electrodes. The second chamber in certain preferred embodiments includes more than one electrode array, for example two electrode arrays on opposite sides of the electrode chamber. Suitable electrode array configurations will be readily apparent to those of ordinary skill in the art, given the benefit of the present disclosure, for example electrode array configurations presented in U.S. Pat. No. 6,277,258, hereby incorporated by reference herein in its entirety for all purposes.

In certain preferred embodiments, each electrode of the array is individually controlled to provide an electric field gradient that can be dynamically controlled (i.e., maintained and adjusted during the course of analyte focusing and/or separation). Control can be manual from the device controller, manually from the device's associated computer, or automatically from the computer once the computer has received feedback from a monitor, such as an optical monitor, for example a video signal, or other suitable monitoring device, following analyte focusing. The controller can sense the electrode's voltage and reset its voltage to its initial setting. Such monitoring allows for computer detection of various peaks, optimization of the separation by locally adjusting the field gradient to tease separated peaks apart, and then pull off those peaks that were selected by the operator either before or during a separation. Suitable configurations of the electrodes, controls, computer equipment and the like will be readily apparent to those of ordinary skill in the art, given the benefit of the present disclosure, for example configurations presented in U.S. Pat. No. 6,277,258, which as noted above is incorporated herein in its entirety for all purposes.

In accordance with certain preferred embodiments of the device and methods, the electronically generated field can take on arbitrary shapes including exponential profiles, steps, and even locally reversed gradients, for example, to elute proteins. The field shape can be monitored and maintained by computer and modified “on-the-fly” on a point-by-point basis, both spatially and temporally. During a run the operator can optimize the local properties of the field to sharpen an individual band, move a band to an offtake port or set up a moving gradient to elute one or more bands from the chamber. With online monitoring, for example optical such as UV/Visible monitoring, or potentiometric monitoring, in place, the operator could be replaced by a computer programmed to detect focused peaks and automatically adjust the field shape to optimize the separation and, when necessary, offload products. Suitable monitoring systems and configurations will be readily apparent to those of ordinary skill in the art, given the benefit of the present disclosure.

The dynamic electric field gradient focusing provided by the methods and devices optionally relies in part on field gradient control, which includes hardware and software Representative gradient control hardware and software are discussed below.

The dynamic electric field gradient focusing provided by the methods and devices optionally relies in part on field gradient control, which includes hardware and software. Representative gradient control hardware and software are discussed below.

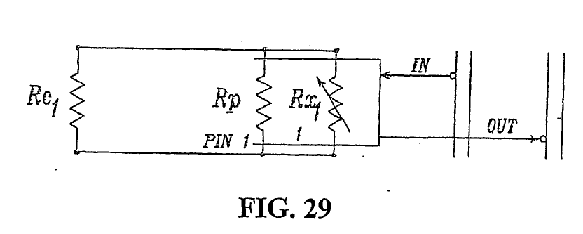

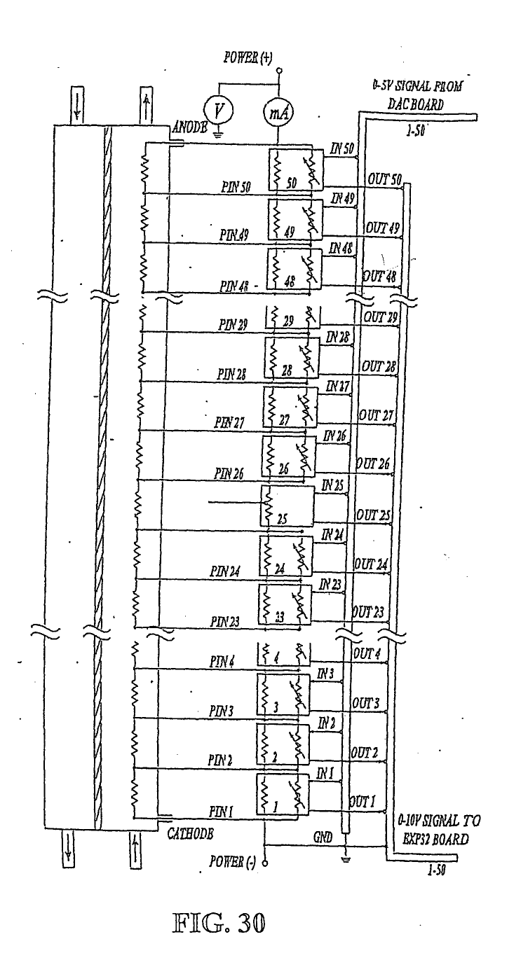

The control circuits are designed to manipulate the field gradient by adjusting the effective electrical resistance between each two adjacent electrodes (see FIG. 29). In one embodiment, each pair of electrodes is connected to one of the 50 controller units. A schematic of such an embodiment is shown in FIG. 30, in which the blocks with dash line frames are controller units and each of the controller units handles the data acquisition and the resistance control of two adjacent electrodes.

The electrical resistance between two adjacent electrodes Rᵢ is determined by the sum of the resistance of three parallel resistors, Rcᵢ, Rpᵢ, and Rxᵢ. Note that the buffer between electrodes is considered as a resistor Rcᵢ.

$${R}_{t} = \frac{{Rc}_{i} \cdot {Rp}_{i} \cdot {Rx}_{i}}{{Rc}_{i} \cdot {Rp}_{i} + {Rc}_{i} \cdot {Rx}_{i} + {Rp}_{i} \cdot {Rx}_{i}} \qquad ( 1 )$$

The resistors Rpᵢ are used for protective purpose and have 1MΩ resistance. Because Rₚ>>Rcᵢ, Rₚ>>Rx₁. Equation (1) can be simplified as

$${R}_{i} = \frac{{Rc}_{i} \cdot {Rx}_{i}}{{Rc}_{i} + {Rx}_{i}} \qquad ( 2 )$$

By changing each Rxᵢ, the circuits adjust each Rᵢ indirectly. By Ohms Law, the potential drop between two electrodes is determined by the resistance between them if the total current going through is constant. The potential drop between the two adjacent electrodes is given by

$${V}_{i} = {V}_{total} \cdot \frac{{R}_{i}}{{\sum }_{i}^{50} {R}_{i}} \qquad ( 3 )$$

Since the field strength is proportional to the potential drop with the electrodes equally spaced, we can manipulate the field strength point by point by adjusting each Rxᵢ, independently.

$${E}_{i} = \frac{{V}_{i}}{d} = \frac{{V}_{total}}{d} \cdot \frac{{R}_{i}}{{\sum }_{i}^{50} {R}_{i}} \qquad ( 4 )$$

where d is the distance between the two adjacent electrodes. An electric field gradient in any shape, linear or nonlinear, continuous or stepwise, can be produced with a limitation to the conductivity of the buffer. Note that the resistance between two parallel-connected resistors is always less than any one of them in other words, Rᵢ<Rcᵢ must be satisfied. There is more than one group of Rᵢ that satisfies Equation 4, in other words, different groups of Rxᵢ can be used to establish the same field gradient with the total current going through the chamber arbitrarily. There is no unique equilibrium state. To solve the problem, a small modification to unit No. 25 is made by disabling its control function and replacing Rp₂₅ with a 5 kΩ resistor. The total current going through the chamber was fixed, and given by

$$I = \frac{{V}_{25} \cdot {Rp}_{25} \cdot {Rc}_{25}}{( {Rp}_{25} + {Rc}_{25} )} \qquad ( 5 )$$

V₂₅ has a unique value for a specific field gradient, and can be calculated from the total potential drop across the chamber. Rcᵢ is determined by the conductivity of the buffer. Therefore, there is a unique value of Rxᵢ that satisfies Equation 4.

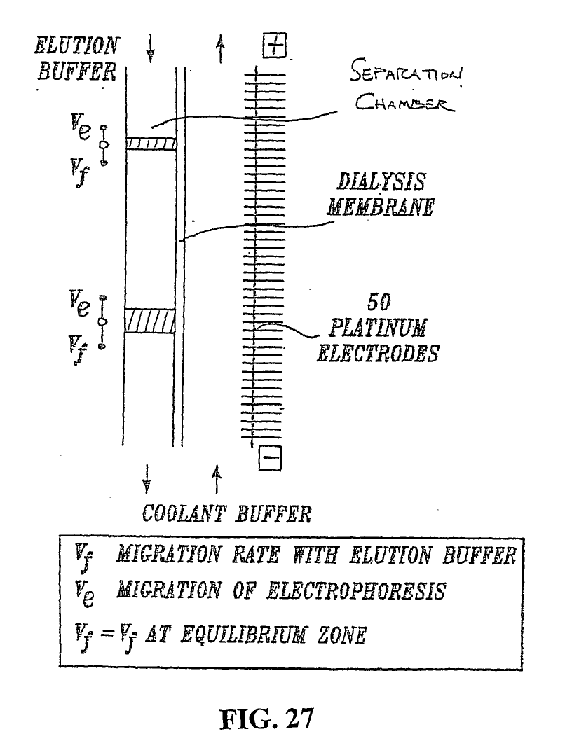

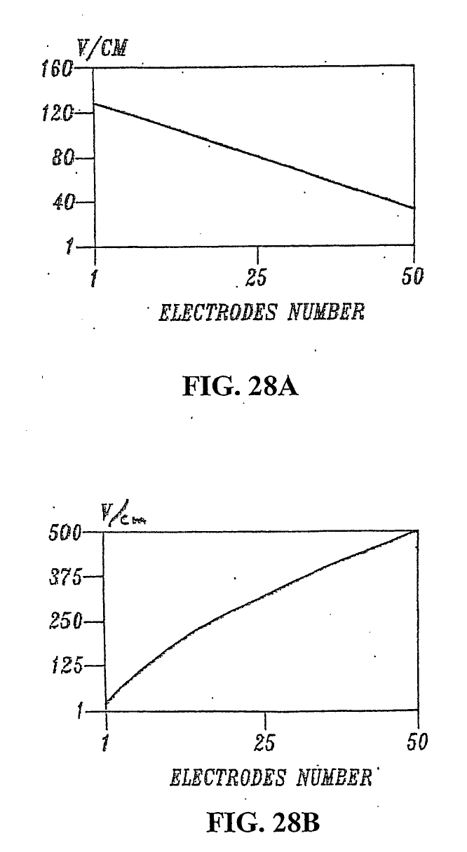

In certain preferred embodiments, dynamic electric field gradients are created by a computer-controlled external circuit, which manipulates the field strength between each pair of adjacent electrodes, as exemplified in FIG. 27. Varying field strength along the separation chamber can thus be achieved. FIGS. 28A and 28B are graphical representations of linear electric field gradients so generated.

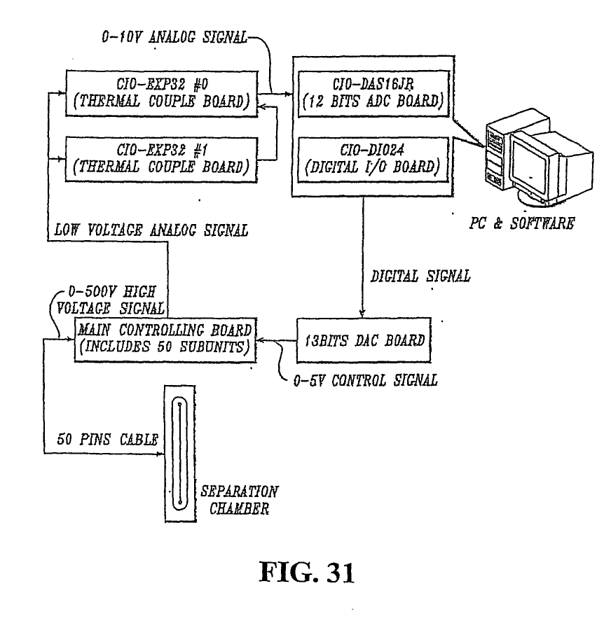

Representative gradient control circuits are shown schematically in FIG. 31. The blocks represent electronic boards, the lines represent standard ribbon cables. Referring to FIG. 31, the PC monitor/controller board and the 13 bit DAC board were built in our laboratory. Some modifications have been made for better performance. The data channels between the two CIO-EXP32 boards and the CIO-DAS16Jr boards are programmed rather than being physically connected. CIO-DAS16Jr and CIO-DIO24 are plugged into extension slots of the PC. The two thermocouple boards CIO-EXP32, the 16-channel ADC board CIO-DAS16/Jr and the 24-channel Digital I/O board CIO-DIO24 were purchased from ComputerBoards, Inc. Standard SCSI ribbon cables are used to connect all the boards. There are 50 controller units plugged into the mother board. Each unit corresponds to one pair of electrodes. The whole system was grounded to protect the circuits from unexpected shock.

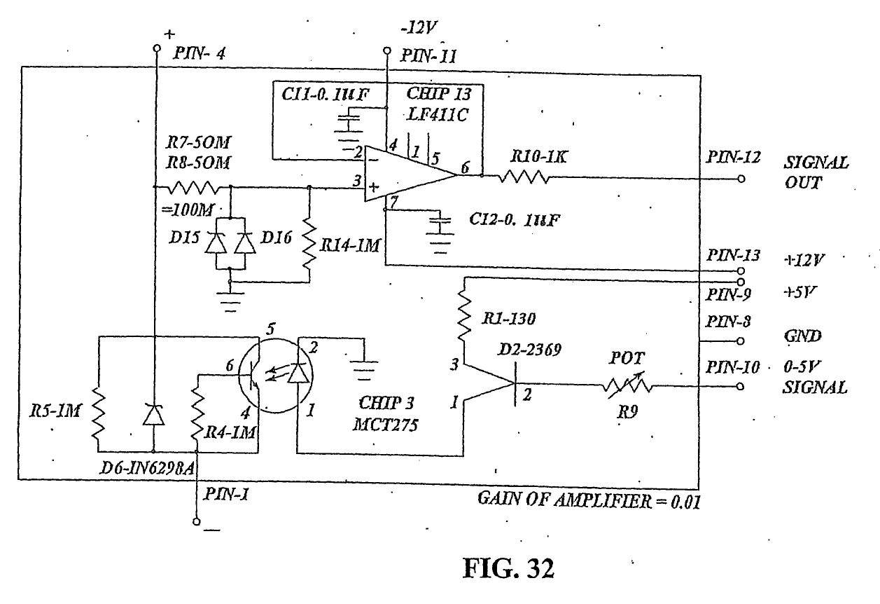

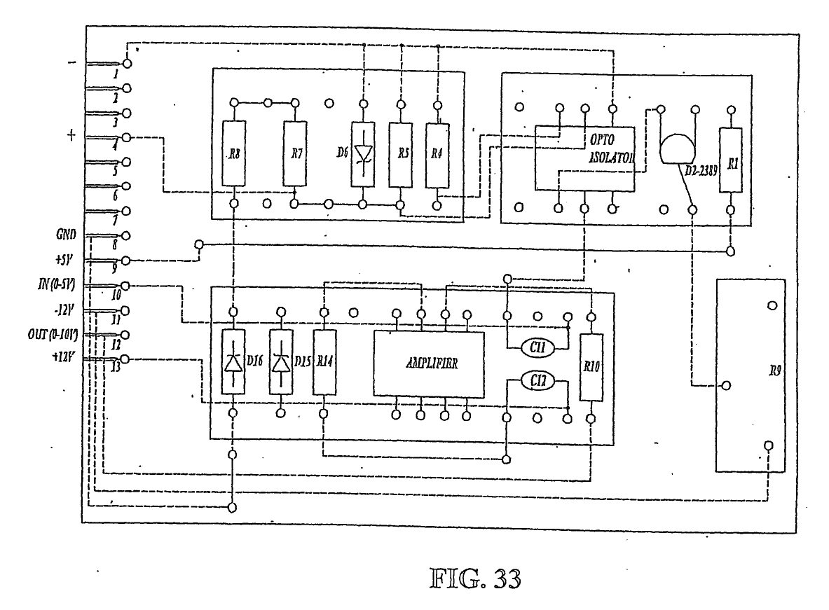

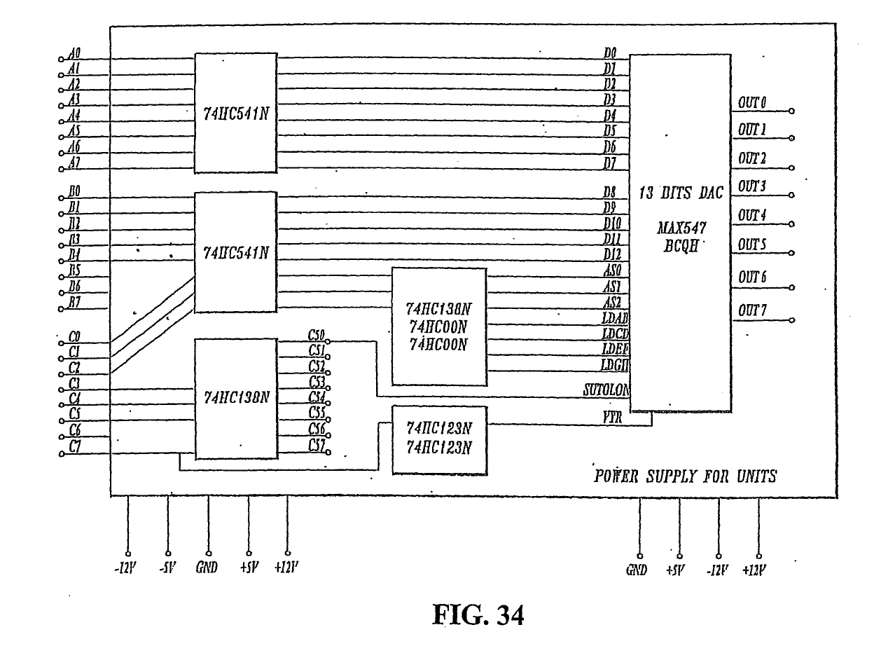

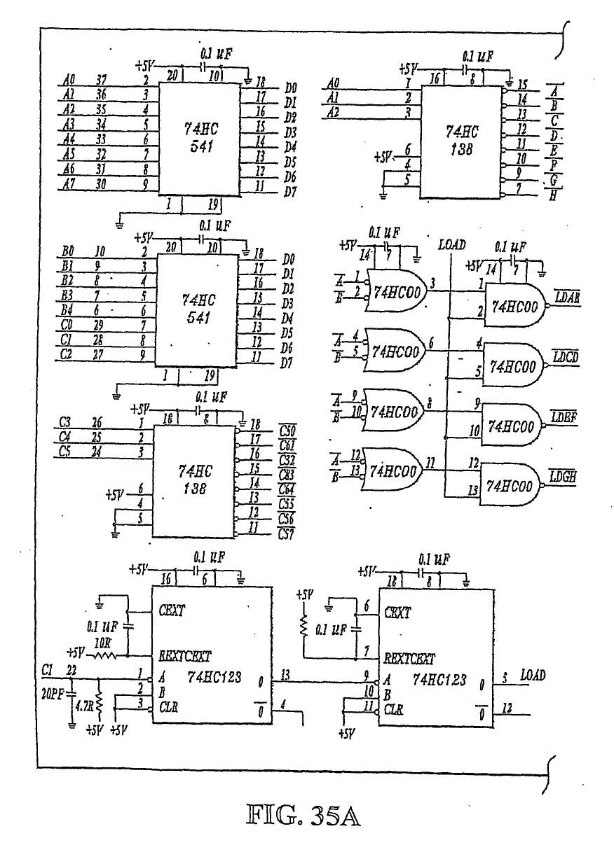

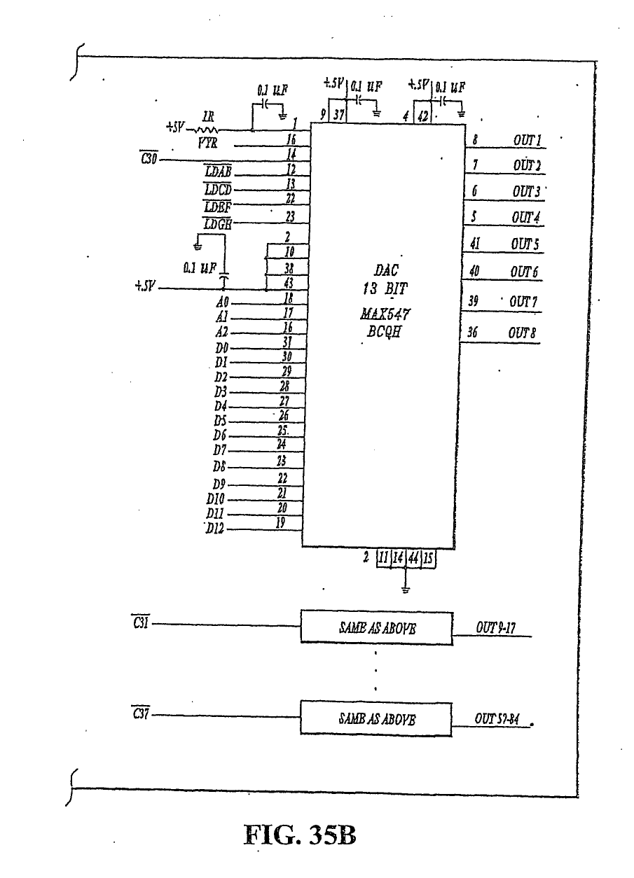

The gradient control is accomplished with PC-controlled circuits, diagrammed in FIG. 32, which are composed of electronic circuit boards. Pin 1 and 4 are connected to electrodes and neighboring units. The electrical potential on the electrode is reduced by 1/100, then enters amplifier LF411C where the load of the signal is increased. The signal is then sent to EXP32 board through pin 12, and the control signal (pin 10, 0-5 V) from the DAC board adjusts the current going through the optical isolator MCT275. A circuit diagram of the controller unit is shown in FIG. 33. A logic diagram for circuit diagram for ADC board is shown in FIG. 34. A circuit diagram for the ADC board with components identified is shown in FIG. 35. The circuits scan all 50 electrodes and scale the signals down by 1/100. Then the signals were sent to ADC board where 0-10V analog signals are digitized. The computer compares these readings with the programmed gradient, then sends its commands in digital signals to DAC board via the Digital I/O boards. In the DAC board, the command signals are converted to 0-5V analog signals, then sent to the 50 units on the PC monitor/controller board. Those units adjust the current going through the units, or we can say change the values of resistance Rxᵢ. Note that the Rxᵢ do not exist physically, and they are the resistance to current going through the chip MCT275, an optically isolated controller. The scan/response cycle for the circuits is set at about 0.5 sec, and could be adjusted by the program. A 600V DC power supply (Xantrex) supplies power to the chamber. The power to all the boards is supplied by the computer. As noted above, the second chamber can include more than one electrode array. For example, two electrode arrays can be associated with a single separation chamber in a configuration in which the separation chamber is positioned in between the two arrays. Similarly, the second (electrode) chamber can include, for example, four arrays positioned about a separation chamber in a quadrupole-type configuration. Other preferred embodiments can include more than one second chamber, each having one or more electrode arrays.

Certain preferred embodiments of the device include, in addition to the electrode array, an electrode pair. In this embodiment, the electrodes of the pair are positioned adjacent opposing ends of the electrode array.

In operation, a first or sample fluid is caused to flow through the separation chamber. Where an electrode chamber is employed, a second or electrode fluid is typically flowed through the electrode chamber. The first and second liquids may be, but need not be, the same. The fluids generally are liquids, and may comprise water or advantageously may comprise buffer. Generally, a higher concentration of buffer stabilizes the protein sample and therefore avoids precipitation. However, in general, high ionic strength means high conductivity of the buffer, which increases the heat generation and power consumption and sets a limit for the highest suitable field strength. Typical field strengths include, for example, 180 to 300 v/cm. Advantageously, the same buffer is used for the first liquid and second liquid, excluding the dissolved gel where a soluble gel is used to ensure the ion balance between the two sides. Advantageously, the device is oriented vertically, that is, so that the flow of fluid through the chambers is substantially vertical, with the buffer in the electrode chamber flowing upward, effectively removing the tiny gas bubbles generated at the electrodes and acting as coolant to remove the Joule heat generated. In certain preferred embodiments, this second liquid is then run through a cooling apparatus, such as a cooling bath, heat exchanger, and the like, to remove the heat from the second liquid and the second liquid is then recycled back into the electrode chamber.

Another important role of the second liquid is to conduct the electric field through the permeable membrane to the separation chamber. Suitable first and second liquids will be readily apparent to those of ordinary skill in the art, given the benefit of this disclosure.

Devices disclosed here are useful in focusing and separating charged analytes. In certain preferred embodiments, the focused analytes can be eluted from the device through one or more additional ports positioned midway along the separation chamber between the inlet port and the outlet port. Basically, the desired analyte can be focused to a region of the chamber from which the analyte can be eluted through a port. Such focusing to a particular location can be brought about, for example, by changing the flow rate of the sample fluid, adjusting the electric field gradient strength, adjusting the electric field gradient shape, changing the pH of the sample fluid, etc. Other suitable means for focusing a band of charged analyte to a particular position within the separation chamber will be readily apparent to one of skill in the art, given the benefit of the present disclosure. Analytes can be eluted from the separation chamber by electric field, pressure, vacuum, or other motive force.

Certain preferred embodiments of the device can further include a monitoring feature which detects analyte migration. Suitable analyte detection includes optical and potentiometric methods. Optical methods include providing a clear window in the first chamber so that an operator can observe the focusing of the bands directly, and further include optical methods such as UV/Visible spectroscopy that can be monitored by the operator or by computer. Optional integration of the signal put forth from the monitoring feature with software allows automation and computer optimization of analyte loading, separation, and elution steps.

The device can be operated in a continuous mode in which analyte for focusing and/or separation is continuously loaded into the separation chamber and focused to offtake ports where the analytes are continuously eluted. In the alternative, the device can be operated in a batch mode in which the analyte is loaded in its entirety and then focused.

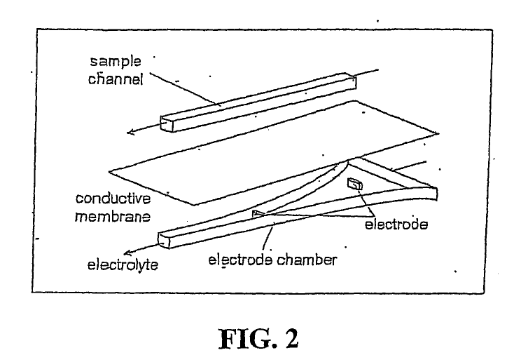

Certain preferred embodiments of the electrophoretic devices comprise a layered assembly in which the separation chamber and the electrode chamber are separated by a porous membrane. The separation chamber is a conduit that may have a shaped geometry, where sample peaks are loaded, held and off-loaded or eluted. The electrode chamber may have a shaped geometry and has at least one built-in electrode pair, where there is one anode and one cathode. Application of a DC voltage to the electrodes results in an electric field, with an intensity inversely proportional to the combined separation chamber and electrode chamber cross-section at a given point. The electric field strength will vary along the axis of flow. To generate a linear electric field gradient through the use of one or more configured chambers, the combined chambers typically will have a hyperbolic shape, but nonlinear fields are possible by selecting the appropriate combined chamber geometry. The magnitude or slope of the field gradient may be manipulated by adjusting the voltage applied to the electrodes. The porous membrane must conductive for the passage of small ionic species and electrical current, thereby communicating the electric field to the separation chamber. The pore size of the membrane is such that all molecules designated as samples will be retained in the sample chamber. A buffer system typically is required for the device to maintain stable pH and provide sufficient conductivity to carry the electrical current throughout the fluidic passages of the electrode chamber and separation chamber. FIG. 2 is an exploded view of an exemplary device comprising a uniform separation chamber and a non-uniform electrode chamber and an electrode pair, as well and demonstrates the representative orientation of device components. The arrow heads indicated the direction of buffer flow. The electric field gradient would cause solute to migrate in the opposite direction to buffer flow. Of course it will be recognized that, in addition to or in lieu of having one or both of the chambers configured, an electrode array can be used to generate or to affect the electric field gradient. Other suitable layered configurations will be readily apparent to those of skill in the art, given the benefit of the present disclosure.

The electric field strength will vary along the axis of flow. As mentioned above, where configured chambers are used to generate the gradient, the combined chambers will have a hyperbolic shape to generate a linear electric field gradient. Nonlinear fields are possible by selecting the appropriate electrode and/or separation chamber geometry. The magnitude or slope of the field gradient may be manipulated by adjusting the voltage applied to the electrodes. The porous membrane must conductive for the passage of small ionic species and electrical current, thereby producing a similar field gradient in the separation chamber. The pore size of the membrane is such that all molecules designated as samples will be retained in the sample chamber. A buffer system typically is required for the device to maintain stable pH and provide sufficient conductivity to carry the electrical current throughout the fluidic passages of the electrode and separation chambers.

In accordance with certain preferred embodiments, the devices disclosed here have a first block comprising a first trough having an inlet for introducing liquid to the first trough and an outlet for exiting liquid from the first trough. A second block has a second trough with an inlet for introducing a second liquid to the second trough and an outlet for exiting the second liquid from the second trough. The second trough further comprises electrodes, optionally an electrode array, positioned in the second trough, wherein the first trough and the second trough are substantially coincident and form a channel when the first block is sealed to the second block. A permeable material is provided intermediate the first and second blocks, dividing the channel formed when the first block is sealed to the second block into a first (separation) chamber and a second (electrode) chamber. The second chamber includes the electrodes. The first chamber holds molecular sieve to shift the location of a stationary focused band of charged analyte in the first chamber for a given set of focusing process parameters. The device as such is in the configuration of a discrete unit, or “chip” or consumable cartridge, for example a microfluidic cartridge, which can be swapped out of a suitable receptacle in a laboratory or processing instrument or the like.

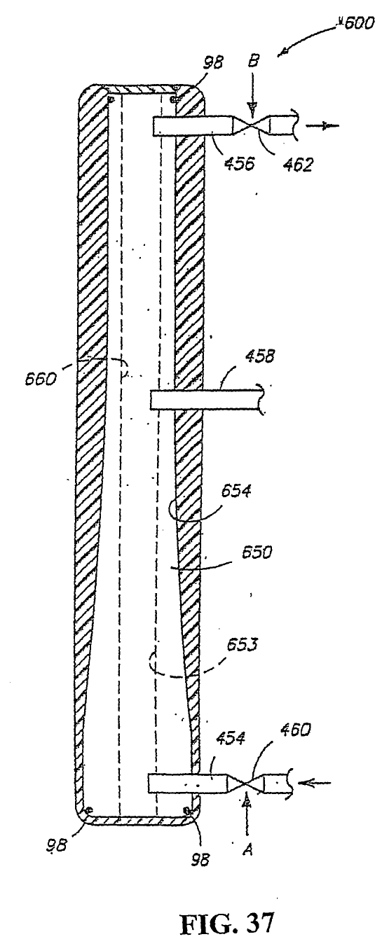

FIG. 37 illustrates another preferred embodiment. Device 600 includes electrode chamber 650 having a varying cross-sectional area defined by electrode chamber walls 654. Positioned within the electrode chamber is a permeable conduit membrane 660, which defines separation chamber 653. The separation chamber 653 is encircled longitudinally by the electrode chamber 650. As illustrated in FIG. 37, permeable membrane 660 is uniform cross-sectionally such that the fluid velocity within the separation chamber is uniform. In certain preferred embodiments the membrane 660, and thereby the separation chamber 653, is tubular, preferably cylindrically tubular. In other preferred embodiments, the separation chamber can be non-tubular, i.e., can be some other geometry, and can be non-uniform cross-sectionally. Membrane 660 is in certain preferred embodiments a dialysis membrane. Sample fluid flows into the separation chamber 653 through inlet port 454, and exits the chamber through outlet port 456. Valves 460 and 462 are provided in-line with inflow and outflow ports 454 and 456, respectively. These valves are operably connected via control signals A and B to a controller, which can be manually controlled or can be computer-controlled as desired. The valves can thus be opened or closed, either fully or partially, to govern the flow rate and volume of fluid introduced into the chamber. An additional port 458 is located intermediate the inlet and outlet ports of the separation chamber and permits the removal or addition of fluid to or from the separation chamber. For example, a focused band of charged analyte could be brought under the additional port and the analyte band could be extracted through the port, for example, by any of the means identified herein. Of course, more than one such additional port could be incorporated. Device 600 further includes electrodes 98, which in certain preferred embodiments are annular, located at proximate ends of the electrode chamber 650, preferably adjacent electrode chamber walls 654 and thus remote from the membrane 660. The converging walls 654 of the electrode chamber 650 cause the current density of electrical current flowing between electrodes 98 to concentrate toward the converging upper end to create a gradient in the electric field within the separation chamber.

In a method aspect, an electrophoretic method for focusing a charged analyte is provided. In the method, a device in accordance with the embodiments above is provided, a first fluid comprising at least one charged analyte is introduced into the separation chamber and an electric field gradient is applied to the charged analyte in the separation chamber to focus the charged analyte in the electric field gradient, wherein the separation chamber contains molecular sieve operative to shift the location at which a stationary focused band of a charged analyte forms under a given set of focusing process parameters. The electric field gradient is preferably generated by an electrode array by individually adjusting the electrode voltages of each element of the array. In certain preferred embodiments, the electric field gradient is dynamically controlled, that is to say the electric field gradient is changed or adjusted while the focusing takes place.

In certain preferred embodiments, a hydrodynamic force is applied to the first fluid by pumping the first fluid through the first chamber. The first fluid typically is a liquid with flow rates ranging, e.g., from 0.1 to 10 μL/min. for analytical applications, and, e.g., from 10 to 200 μL/min. for preparative applications. The flow rate is chosen to provide the desired separation, in other words so that the hydrodynamic force, when combined with the effect of the molecular sieve, counters the electric field gradient at a position between the weakest and the strongest part of the electric field. In this fashion, the analyte will be retained within the first chamber. Factors that affect the choice of flow rate include, for example, the viscosity and density of the liquid, strength of the electric field gradient, net charge of the analyte, etc. Suitable flow rates will depend, therefore, in part upon the electric field gradient that is chosen. Suitable flow rates can be readily determined by routine trial and error.

Certain preferred embodiments of the devices and methods herein are suited for focusing and separating charged analytes. Charged analytes that can be focused include, e.g., charged polymers, carbohydrates, and biological analytes, such as proteins, peptides, oligonucleotides, polynucleotides, hormones, biomarkers, and the like, and mixtures of any of these. In particular, charged analytes which have similar charge to mass ratios, such as DNA, RNA, etc., can be separated and focused on the basis of differences in their respective molecular weights. Absent the presence of the molecular sieve of the current devices and methods, such analytes are difficult to fully separate due to the similar charge to mass ratios.

Materials with little or no net charge sorbed into charged carriers, for example micelles and liposomes, can also be focused and separated with the device. For example, proteins that exhibit little net charge can be sorbed into a charged carrier such that the protein acquires the charge of the charged carrier. In certain preferred embodiments, a detergent, for example sodium dodecyl sulfate (SDS), is used as the charged carrier. Without wishing to be bound to a theory, it is presently believed that the SDS binds strongly to protein molecules and “unfolds” them into semi-rigid rods whose lengths are proportional to the length of the polypeptide chain, and hence approximately proportional to molecular weight. Because of the magnitude of the charge of the bound detergent molecules, the protein complexed with such a detergent takes on a high net charge. Furthermore, the total charge is approximately proportional to molecular weight, as the detergent's charge vastly exceeds the protein's own intrinsic charge. Thus, the charge per unit length of a protein-SDS complex is essentially independent of molecular weight. This feature gives protein-SDS complexes essentially equal electrophoretic mobility in a non-restrictive medium. Separation and focusing is then brought about by the molecular sieve (acting in conjunction with the other focusing process parameters) on the basis of the molecular weights of the protein-SDS complexes.

The separation chamber in any of the above-disclosed embodiments can either be an open channel or can be packed with a media, such as a gel or granular packing, to reduce the convective dispersion and help maintain sharp peaks. In certain preferred embodiments, the separation chamber contains a fluid medium. Suitable fluid media include simple fluids such as, for example, buffered water. Also included are complex fluids, for example, a water/acetonitrile/methanol mixture, or polymer solutions such as, for example, linear polyacrylamide, polyvinyl alcohol, methyl cellulose solutions and the like. The fluid media in certain preferred embodiments further comprises a chromatography support medium or packing. Suitable packings can be of any size or type provided that the solute being focused does not irreversibly bind to the packing. Suitable packings include porous and nonporous, pellicular and tentacle, glass, plastic, ceramic, and any nonconductor or semiconductor. Other suitable packings include ion-exchange, affinity, reverse phase size exclusion, gel filtration and hyperbolic interaction supports.

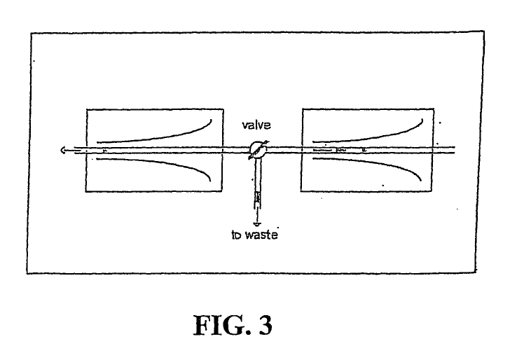

In accordance with certain preferred embodiments, a “notch filte” for sample peaks can be constructed from two serial connected devices and a switching valve. Sample peaks within certain mobility range can be selectively cut from a series of peaks, routed to a second device and prepared for subsequent analysis. FIG. 3 depicts the configuration for an exemplary “notch filter” embodiment of the systems disclosed here. It can be seen that two devices are connected in a serial fashion.

The electrophoretic devices can be constructed of any suitable material that is compatible with either aqueous or organic solvents, or both, depending on the intended application and environment of use. Exemplary materials include PEEK, TEFLON, acrylic, etc. Thus, for example, the separation chamber can be etched or otherwise formed as a groove or channel in a substrate of such material. The membrane seated against the substrate completes the separation chamber, leaving the ends open (preferably valved) for flow. The device may be constructed of material that is optically clear over the UV-Vis-IR spectral range to permit imaging or detection of isolated molecules in the chamber. This may include UV transparent acrylic, quartz, TEFLON AF, etc.

Detection strategies for such devices may also include monitoring the entering and exiting streams for tracking of materials. These would be considered point detectors. If the device is constructed of optically clear material, as mentioned above, point detectors could be spatially distributed along the length of the chamber, as opposed to imaging the entire chamber. Point detectors may be distributed throughout the system for overall tracking of material (e.g., at the exit of a switching valve to monitor routing of peaks).

As disclosed and described above, the electrophoresis device uses a porous, conductive layer to separate the electrode chamber from the focusing chamber. The porous layer may be a dialysis membrane, ceramic membrane or other porous material that allows conduction of ions and electrical current. The molecular weight cut-off (MWCO) for the porous layer may range from 100-30,000 MW. Typically, small molecule applications may require a porous layer having a 100-200 MWCO and proteins applications may require a porous layer having a MWCO>1000.

In operation, the device includes the flow of a first fluid, typically a liquid, through the first, or separation chamber and the flow of a second fluid, also typically a liquid, through the second, or electrode chamber. Generally, the first liquid is an electrophoretic eluant (e.g., buffer solution) and the second liquid is a coolant. Suitable liquids include simple liquids such as buffered water, complex fluids, for example mixtures of water and solvent, etc. The first liquid can be the same as or different from the second liquid. During focusing and separation, and depending on the requirements of the particular separation, the composition of either the first and/or the second liquid can be changed to achieve the desired result. As noted above, liquid flow through the separation chamber preferably opposes the direction of electrophoretic migration of the analyte and can be driven by any one of a variety of forces including electric field, pressure, vacuum, or other motive force. In a preferred embodiment, the direction of liquid flow through the separation chamber is opposite that through the electrode chamber.

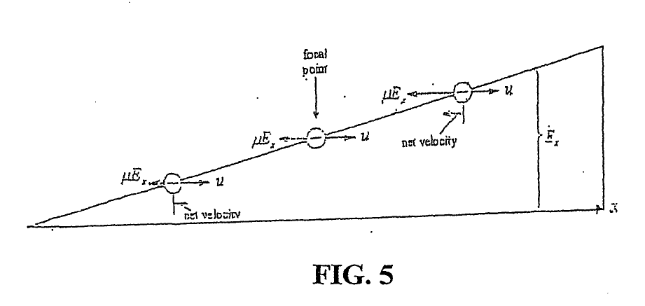

FIG. 5 is a further illustration of electric field gradient focusing. In FIG. 5 a charged solute is pushed from left to right by a chromatographic flow. The electric field will impose an electrophoretic migration velocity proportional to the mobility of the solute. At the point where the elution and migration velocities balance is considered the equilibrium focal point for a solute.

EFGF enables focusing of target molecules at pH values distant from their isoelectric points (pI) and in simple buffer systems. Therefore, EFGF has a distinct advantage over isoelectric focusing (IEF), which has the following inherent limitations: many solutes have low solubilities at their isoelectric points, entire classes of solutes cannot be focused by this method either because they degrade at their isoelectric point (pI), e.g., nucleic acids, or they do not have a readily accessible pI, e.g., polystyrene latexes, and the use of ampholytes for generating the pH gradient can increase the cost per separation substantially. Compared to chromatographic techniques, the equilibrium focusing technologies allow a sample to be “held-in-place”, rather than flow-thru elution, providing a method to collect peaks from multiple injections or trials without having to manually combine collected fractions. Furthermore, the chamber configurations for EFGF allow the separation conditions (e.g., field gradient, buffer composition, pH) to be altered in situ, providing a means to adjust separation resolution or evaluate behavior of target analytes in a changing environment. There is additional benefit of the device with use of an electric field gradient and optionally a hydrodynamic force gradient as a separation driving force, which promotes flexibility for target molecule elution from the chamber. The slope of the gradient can be decreased in a stepwise fashion to selectively release solutes “trapped” within a “mobility window” (i.e., the field strength of the lower gradient setting is insufficient to retain molecules having electrophoretic mobilities within the “step”). Since the holding force on the sample peaks is an electrophoretic migration, devices in accordance with those presented herein will act on charged molecules or any neutral molecules labeled with charged groups or modified to possess an apparent charge. Given that the majority of DNA, proteins, and other small molecules (e.g., metabolites) are charged in an aqueous environment, these devices will have application to separation or management of a very broad range of biological samples. In general, the field gradient focusing technologies have been described as providing separation strategies that are orthogonal to IEF and various chromatographic techniques, therefore these devices and methods may provide researchers with an attractive alternative to LC and PAGE for separation of complex mixtures, as well as providing a new methodology for instrument hyphenation.

In accordance with certain preferred embodiments, the features, attributes, and benefits that can be provided by a SPUR-type device include:

Sample concentration or preconcentration into small microliter volumes

In-line buffer exchange

Capability to capture peaks from multiple trials

Alternative mode of action for separations

Simple buffer systems

Broad range of analytes and application for separations

Operates as stand-alone or integrated component

Small device footprint

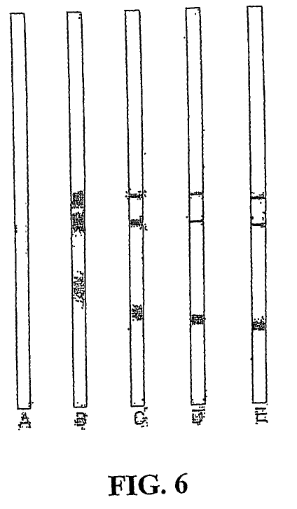

The inherent simplicity of preferred embodiments, e.g., as illustrated in FIG. 2, is advantageous in many applications. Referring again to FIG. 2, the conductive layer (e.g., a dialysis membrane) separates the separation chamber from the electrode chamber, which is tailored with a hyperbolic curvature to form a linear field gradient. The conductive layer should allow passage of buffer ions and electric current, but should have pore structure that restricts translocation of target molecules from the focusing channel. FIG. 6 presents a series of images extracted from a simulation of a focusing system where three proteins have been injected as a dilute, homogenous mixture. According to model results, a high mobility molecule (small or strongly charged) moves fast to its equilibrium point. Therefore, a focused band can be established in a relatively short amount of time. For a low mobility molecule (big and weakly charged), focusing to equilibrium occurs on a longer time scale. As an example, with field strengths ranging from 20-200 volt/cm, at opposite ends of the chamber, a molecule with an electrophoretic mobility of 5×10-5 cm2/volt.sec can focus into a 2 mm band in approximate 12 minutes. While, a molecule with a mobility of 5×10-6 cm2/volt.sec will reach its focal point in approximately 2 hrs. Slower moving analytes create a challenge for equilibrium focusing techniques, but an increase in focusing speed may be achieved at higher flow rates and higher field strengths. Since it is relatively easy to increase the system flow rate, extension of the operational range for such devices primarily focuses on increasing the field strength. The limiting factor to operational conditions may be joule heating and subsequent heat dissipation. It is presently understood that small-scale devices will be capable of 200 V/cm in a 20 mM Tris-phosphate buffer. These field strengths are similar to those used in conventional capillary-scale instruments. It would be advantageous to focus at higher field strengths, but it is believed that fields of 250 V/cm may yield uncontrollable temperature effects and 500 V/cm may prove unrealistic at this scale in the

FIG. 6 shows results of a simulation showing the focusing of a protein mixture. A) A faint smear can be seen near the mid-point of the column as two high mobility proteins begin to focus. B) Three distinct bands, representing R-phycoerythrin (pink), phycocyanin (blue), and myoglobin (brown), are observed at 2.5 min after applying the field gradient. C) and D) show the bands becoming more concentrated at 5 and 7.5 min into the focusing trial. E) Elution of the bands is initiated resulting in a shift downward towards the focusing chamber exit.

A general hyperbolic channel shape for the bottom layer of certain preferred embodiments comprising a configured electrode chamber would generate field strengths ranging from 20 to 200 V/cm at the widest and most narrow points of the channel, respectively, at an applied voltage of 300 V between a single electrode pair. Although a linear field gradient may be preferred for general focusing application, the field shape is not constrained to linear. As a matter of fact, the shape of the configured chamber can be tailored to create a nonlinear field to address specific needs, but a linear gradient is the most universal shape and would typically be the starting point for a separation.

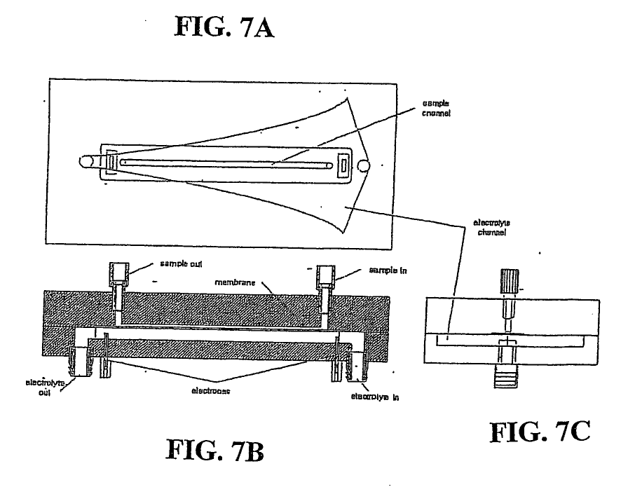

An exemplary device of certain preferred embodiments is illustrated in FIG. 7. Since the cross section of the electrode chamber is significantly larger than the separation chamber, the field gradient is primarily determined by the shape of the electrode chamber. Electrolyte sweeps through the electrode chamber to remove electrolysis products and joule heat. The separation chamber may be packed with a chromatographic media to stabilize convective perturbations. The device is seen to be an assembly of three functional layers including the separation chamber (upper most layer), the “conductive” membrane, and the electrode chamber (lower most layer). The two chamber layers may be fabricated from common plastics (e.g., acrylic or PEEK with TEFLON AF or quartz components) to allow visualization of the separation processes, however chemical compatibility will be a consideration in material selection. The electrodes, housed in the lower layer, are single electrode elements consisting of either gold or platinum metal to prevent hydrolysis-induced breakdown. Trade-offs in the focusing systems have been observed in balancing the operational range and resolution against the ability to dissipate heat.

The range of proposed electrophoretic mobilities accessible by the devices and methods disclosed herein includes a wide range of the peptides that may be encountered when peptide mapping, thereby expanding the application base of the device, method and systems disclosed here.

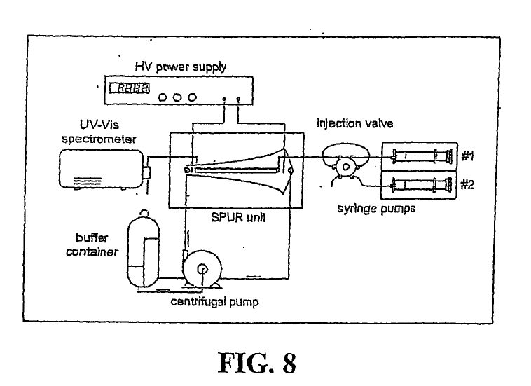

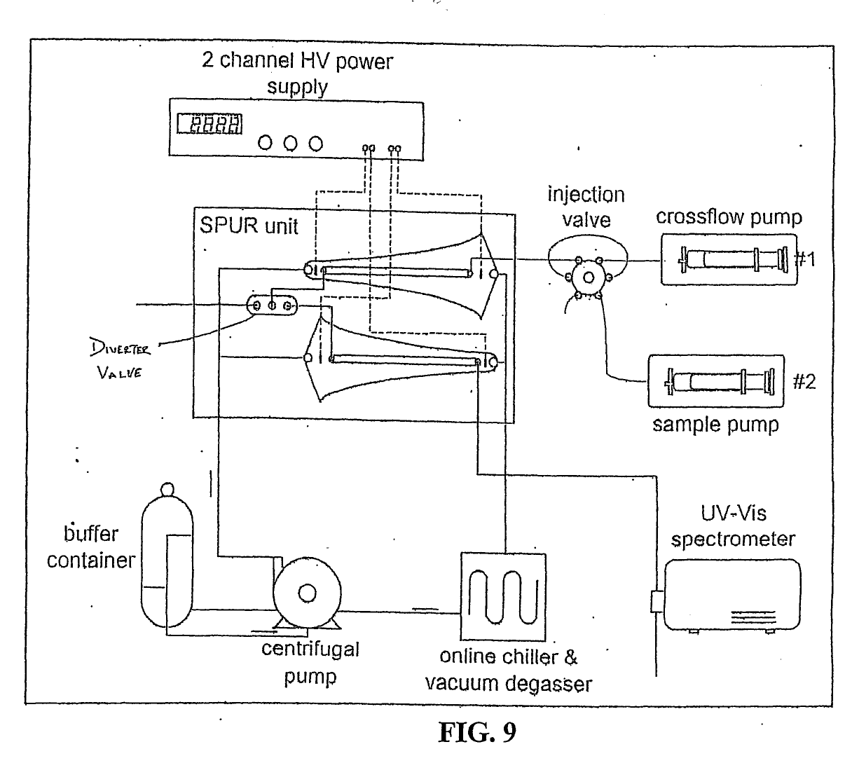

FIGS. 8 and 9 are schematic illustrations showing exemplary apparatuses, with FIG. 8 representing a single electrophoretic device and FIG. 9 representing an apparatus containing two electrophoretic devices. The devices can be controlled via RS-232, LAN, or contact closure interfaces and advantageously can be used with commercially available liquid handlers to allow unattended analyte preconcentration. Fluidic samples, for example, aqueous-phase samples, are injected into the first analytical or separation chamber with the trapping electric field turned on, and the sample is allowed to separate and focus into one or more bands of focused analyte. The electric field is then lowered to allow a low mobility band of analyte to exit or elute from the chamber. As exemplified in FIG. 8, the eluted band then passes to a suitable detector, here a UV-Vis spectrometer, for detection and quantification, and then exits to pass into any other desired sample treatment or detection apparatus. As exemplified in FIG. 9, eluted bands may be passed from the first electrophoretic chamber into a second electrophoretic chamber. In this fashion, bands can be located within either chamber by separately manipulating the strength of the electric field in each chamber. Such a design is advantageous in that it permits the removal of bands of intermediate mobility while allowing the apparatus to retain bands of higher and lower mobility in the electrophoretic chambers. For example, desired bands of low mobility can be eluted from the first chamber and permitted to flow into the second chamber while retaining the remaining bands on the first chamber. Subsequently, the undesired intermediate bands can be eluted from the first chamber and diverted, for example by means of a diverter valve, out of the device, for example into a separate detection and/or sample treatment apparatus or to a waste port. The remaining bands of high mobility can then be flowed into the second chamber and then to the detection/treatment systems as desired. Typical operating parameters of an apparatus of this type with a 1-inch chamber are shown in Table 1:

| | | |
|---|---|---|
| Sample Amount | 10 | micrograms total load |
| Focusing Time | 10 | minutes |
| PH Range | 3-9, | programmable |
| Temp. Range | 10-25° | C. |
| Number of Electrodes | 2 | |
| Eluent Flowrate | 1 | μL/min. |
| Buffer Flowrate | 1 | mL/min. |
| Maximum Voltage | 350 | Volts |
| Maximum Current | 45 | mA |
| Maximum Field Strength | 200 | Volts/cm |

In each of FIG. 8 and FIG. 9, syringe pump #2 represents a device for the introduction of sample to the apparatus. It will be understood that such a device may comprise means for the introduction of free-standing sample, for example a syringe, or may instead comprise the output of an upstream instrument. Further, the UV-Vis spectrophotometer can be replaced by or be followed by any suitable downstream instrument or other sample detection, treatment, or collection device. In this fashion, the apparatus can be used to link up separate instruments in a hyphenated fashion, whereby the sample flows directly from one instrument into the apparatus and then into the next instrument. Additional injections may be used in certain preferred embodiments to accumulate or concentrate low abundance materials while holding previous samples in either the first or, where one is present, the second chamber. Alternatively, continuous flow of sample may be so used, or a combination of continuous flow and additional injections. Further, additional such electrophoretic devices may be used in serial or in parallel networks to provide additional separation flexibility for accumulating multiple analytes for collection or analysis. Additional peripherals may be added for any desired follow-on sample analysis, treatment, collection and the like. Other suitable apparatus designs will be readily apparent to those of skill in the art, given the benefit of this disclosure.

In certain preferred embodiments, either or both of the separation chamber and electrode chamber comprises cartridge-like inserts that are capable of being easily removed and replaced. The chamber inserts typically reside between an inlet and an outlet for flowing a fluid into and out of the insert chamber. The shape of the chamber is determined by the configuration of the insert. Such a configuration is particularly advantageous in that the configured chamber can be swapped out for chambers of different configurations, making a variety of electric field gradient shapes and strengths available in a single instrument. In other preferred embodiments, the electrode chamber comprises a cartridge-like insert that can be swapped out, for example, to permit changing between a non-configured separation chamber and a configured separation chamber. Typically, the electrode chamber insert will comprise the electrodes. In yet other preferred embodiments, the entire device is contained in an insert that is insertable into an instrument properly set up with appropriate fluidic, electric and other necessary connections. Suitable cartridge configurations will be readily apparent to those of skill in the art, given the benefit of the present disclosure.

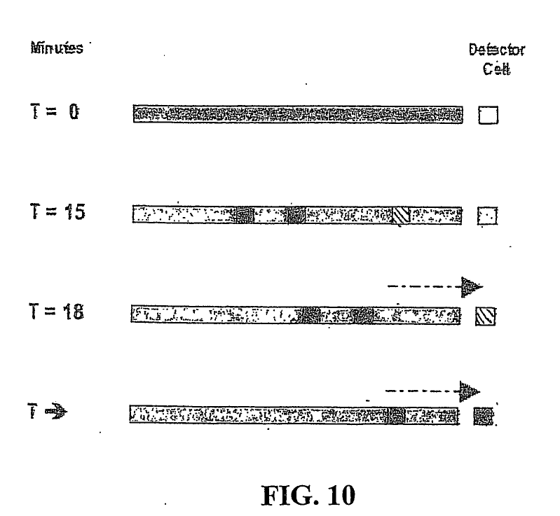

FIG. 10 presents a series of images extracted from a simulation of a focusing system, in which three analytes have been injected as a dilute, homogenous mixture, that illustrate a usage of a device in accordance with the disclosure herein to separate, retain, concentrate, and elute analytes. At the outset, sample is loaded into the device and the electric field is applied. At 15 minutes, the sample has separated and focused into three separate analyte bands, each of which may contain more than one species of analyte. The electric field is then lowered, here at 18 minutes, reducing the electrophoretic force that counters the chromatographic force to an extent sufficient to elute the band of analyte that has the lowest mobility to any suitable downstream detector or further sample treatment device, in the case of FIG. 9 into an optical flowcell for detection and quantification. Advantageously, the remaining bands of interest can be retained in the electrophoretic device by maintaining the electric field at the lower power level while the first band is analyzed. Each of the remaining peaks can then be eluted in the same fashion into the same downstream detector or further sample treatment device, or can be diverted into any other appropriate downstream device. Further, if desired, following elution of the band of lowest mobility additional sample can be loaded into the electrophoretic device for concentrating the remaining species; the two retained species will concentrate while the eluted species will not be retained in the device. As such, the device can be used to purify as well as to concentrate the species of interest. Other suitable applications of the device will be readily apparent to those of skill in the art, given the benefit of the present disclosure.

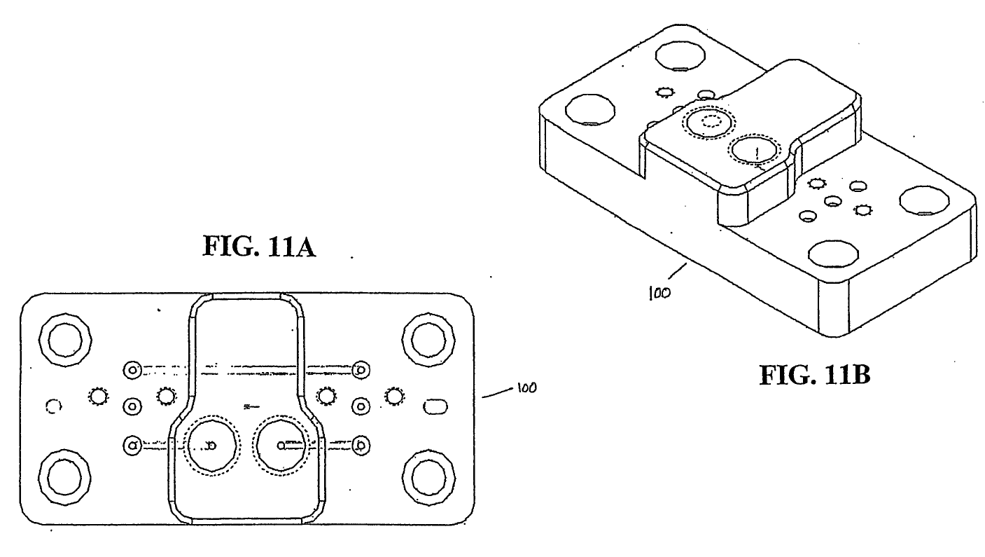

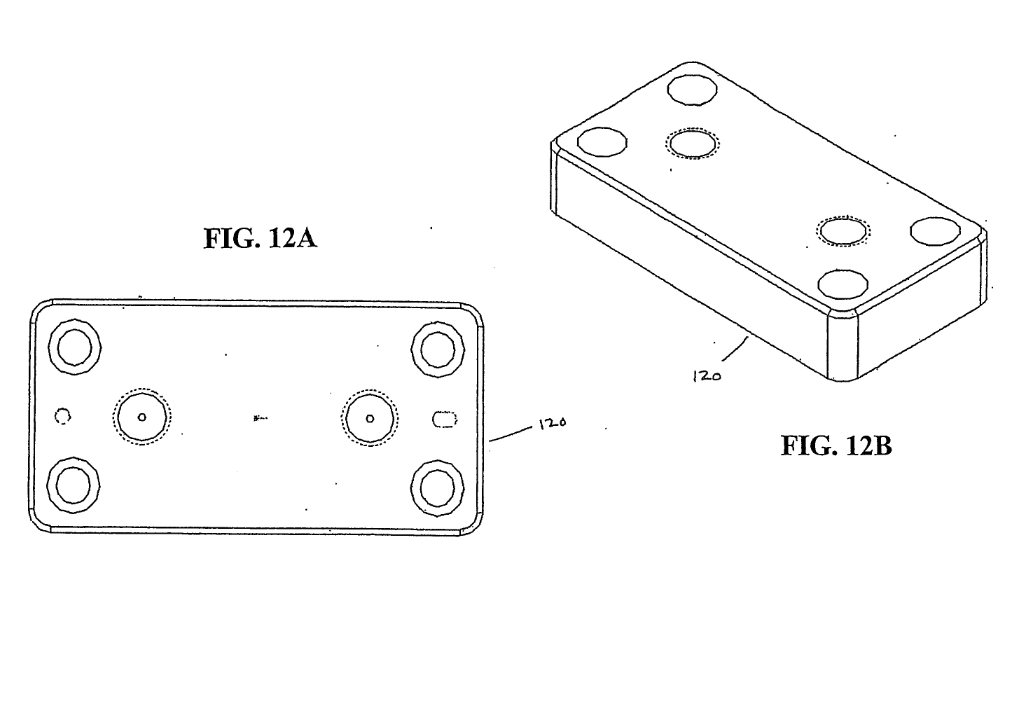

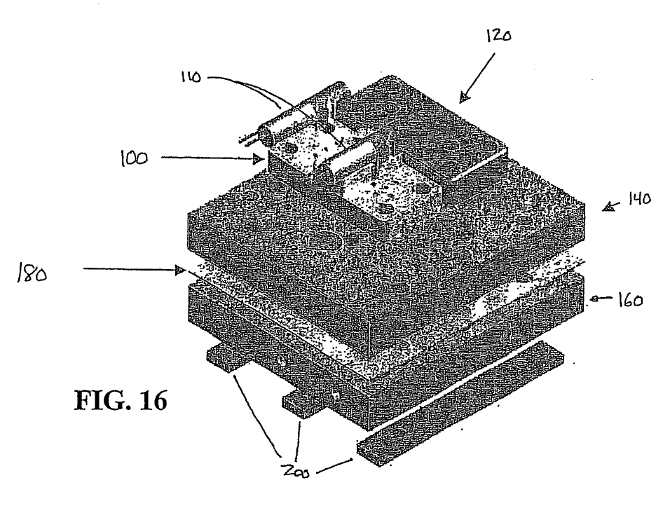

FIGS. 11-16 illustrate a preferred electrophoretic system incorporating two devices in accordance with the disclosure herein, at least one of which incorporates molecular sieve in the separation chamber. The device is comprised of a series of blocks which incorporate the necessary fluid flow paths and electrode paths to make up the device. The overall make-up of the blocks is illustrated in FIG. 16, which also includes a series of nut plate fasteners 200 for transmitting the closing or sealing force of a series of nuts and bolts, not shown, more evenly across the blocks. FIGS. 11A and 11B illustrate top and isometric views of a diverter manifold 100, which as shown in FIG. 16 incorporates one or more diverter valves 110, such as, for example, 3-way valves which are commercially available. Optionally, a multiport diverter valve can be employed, such that a sample can be flowed into the second separation channel or can be diverted to more than one possible additional sample treatment and/or detection system. FIGS. 12A and 12B are top and isometric views of a general fluidic interface manifold 120. While the general fluidic interface manifold presented in this figure directs fluid flow uninterrupted through the manifold, the general fluidic interface manifold can include additional fluid handling devices, such as, for example, one or more diverter valves, one or more sample splitters to distribute a single entering fluid into two or more separate fluidic pathways, etc. Similarly, the diverter manifold may incorporate additional fluid handling devices. Optionally, a single manifold is used which incorporates all of the necessary or desired fluid handling devices. In other preferred embodiments, more than two manifolds are utilized, such as, for example, where more than one separation chamber is incorporated into a single device. Suitable manifold arrangements and fluid handling arrangements of the manifolds will be readily apparent to those of skill in the art, given the benefit of the present disclosure.

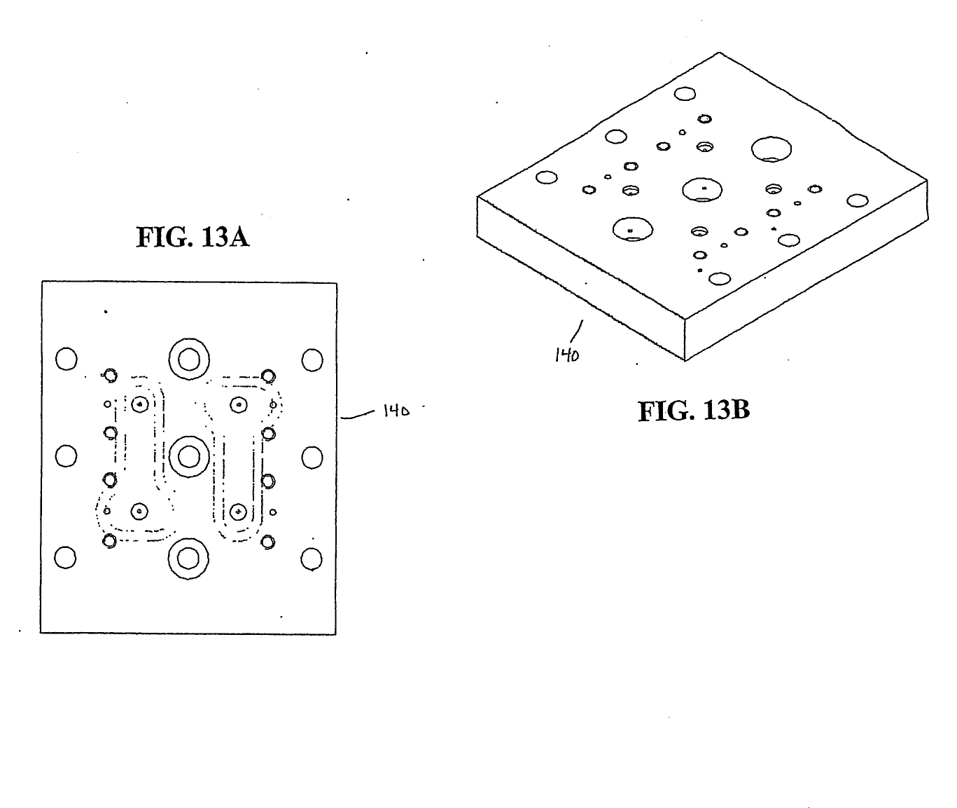

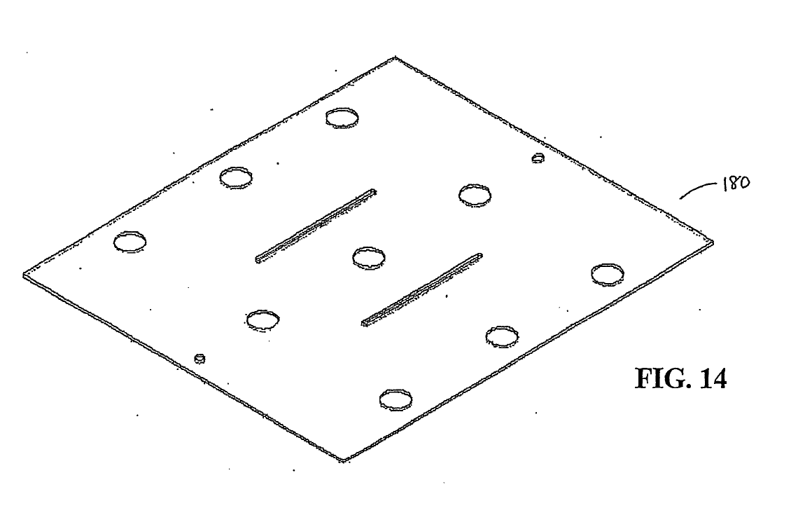

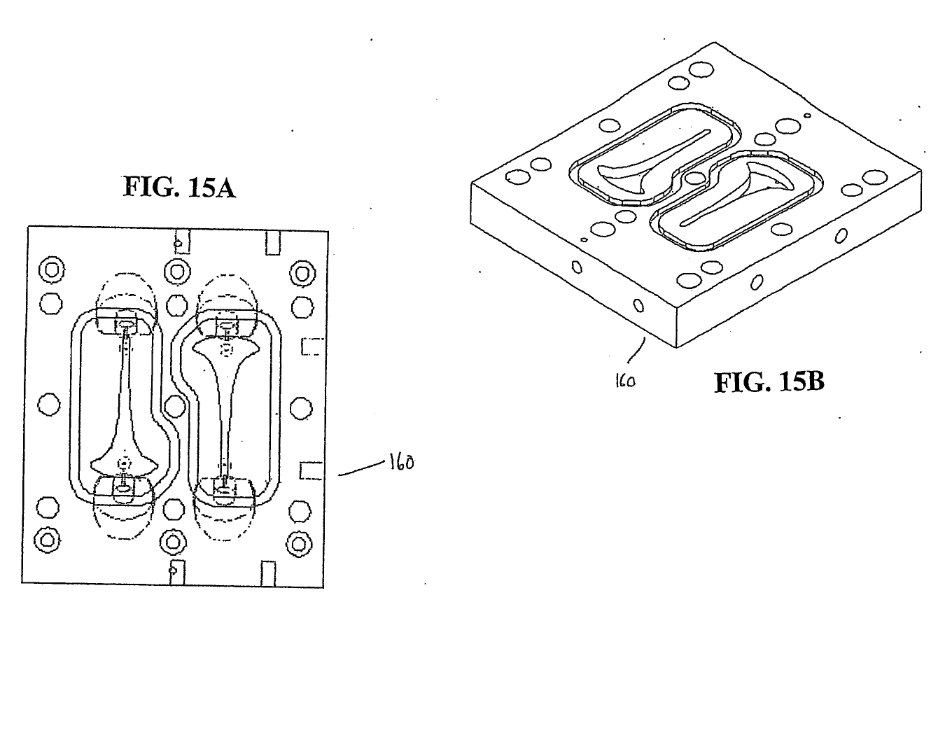

FIGS. 13A and 13B show a top block 140 and FIGS. 15A and 15B show a bottom block 160 of a preferred device. Each of these blocks optionally contains holes for acceptance of a closing mechanism, for example, a nut and bolt configuration which clamps the blocks together and seals the fluidic pathways within the blocks. Sandwiched in between the top and bottom blocks in certain preferred embodiments is a separation channel layer 180, which preferably also serves as a sealing gasket for sealing the top and bottom blocks, an embodiment of which is illustrated in FIG. 14. This separation channel layer may likewise has holes for acceptance of a closing mechanism. The separation channel layer has separation channels cut into and optionally through the layer material, such that the material acts as the side walls and the lower face of the top block serves as the top wall of the separation channels. Alternatively, separation channels may be cut into the lower face of the top plate itself. Other suitable separation channel configurations will be readily apparent to one skilled in the art, given the benefit of the present disclosure.

The top block 140 has fluid pathways cut into the substrate to allow the fluid containing the sample to enter the separation channels. Similarly, the bottom block 160 has fluid pathways for the introduction and removal of fluid into and from the electrode chambers, which are incorporated into the bottom block. The electrode chambers may be cut, carved, stamped, etched or otherwise incorporated into the substrate of the bottom block Alternatively, the bottom block may comprise layers, laminates or composites, in which the electrode chambers are cut out of a first layer such that the first layer acts as the side walls, and the first layer then overlayed a second flat layer, which acts as the bottom wall of the chamber. The latter is advantageous in that a uniform second layer could be mass-produced, and alternative first layers could be custom cut to incorporate different electrode chamber configurations and, therefore, different electric field gradients. The bottom block will further comprise electrode pathways for admitting an electrode into the electrode chamber, typically two electrode pathways per electrode chamber for admitting two electrodes into each electrode chamber. Optionally, an array of electrode pathways will exist for the admission of an array of electrodes into the electrode chamber. A porous, conductive membrane will be interposed between the separation chambers and the electrode chambers to separate the separation channels and the electrode chambers while allowing the electric field gradient of the electrode chambers to be passed through to the separation channels. Such a membrane will typically be a flat material sandwiched between the separation channel layer and the bottom block. Other suitable top and bottom block configurations will be readily apparent to one skilled in the art, given the benefit of this disclosure.

In certain preferred embodiments, an optical sensor flow cell is located between the top block and the manifold block or blocks for detecting and optionally quantifying analyte located in the separated focused bands. The optical sensor flow cell may comprise a block similar to the other blocks in that it may contain holes for acceptance of a closing or sealing mechanism, and may comprise a composite, laminate or other layered configuration. The optical sensor flow cell will typically have a fluid input and output and will have a pair of windows configured such that a beam of light can pass into the flow cell, through the sample, and out of the flow cell. Windows as used herein refers to construction and material selection such that a desired wavelength of light, typically ultraviolet or visible light, can pass through the window in sufficient quantity to allow the light beam to be used as a detector and optionally to allow for quantification by means of the light beam. Advantageously, the window is comprised of a material that permits sufficient light to pass over a range of wavelengths, for example over the entire visible spectrum, preferably over the UV and visible spectrum, to permit a variety of wavelengths of light to pass, to permit detection and analysis over a range of wavelengths. Other suitable optical sensor flow cell configurations include those enabling detection by refraction, fluorescence or phosphorescence and other optical detection means. Such optical detection means are known and will be readily apparent to those of skill in the art, given the benefit of this disclosure. Other preferred embodiments incorporate a detector flow cell, preferably located between the top block and the manifold(s) that incorporate other suitable sensors such as, for example, electrochemical sensors. Other suitable sensors will be readily apparent to those skilled in the art, given the benefit of the present disclosure.

In a typical dual-channel series arrangement, as exemplified in FIGS. 11-16, the fluid containing the sample will flow into the general fluidic interface manifold, then into a fluidic pathway in the top block leading to one end of the first separation channel, typically to the end containing the lowest absolute electric field strength of the gradient. The sample will focus in the separation channel into one or more separate bands, which will simultaneously be held in the channel. At the same time, any species in the sample of insufficient electrophoretic mobility, given the electric field strength being applied, will elute from the channel. After focusing, the electric field strength can be lowered such that the band of lowest electrophoretic mobility elutes from the first separation channel. The band then flows through the top block into the diverter manifold, where it is either directed towards the second separation channel or is diverted to an outlet, for example a waste outlet. Optionally, the band is diverted to an outlet that is attached to a downstream element such as, for example, a further sample treatment or detection instrument or other suitable device, or optionally the band is sent to an outlet for collection. When the band is directed to the second separation channel, it is then held in the second separation channel by the second electric field and possibly separated into further discreet bands. By decreasing the electric field strength in the second separation channel, the band can be eluted from the second channel, from which it passes back up through the general fluidic interface manifold and on to the next downstream element. Optionally, the general fluidic interface manifold comprises one or more diverter valves such that the eluting band can be diverted to one of multiple downstream elements. Such is advantageous, for example, when multiple desired bands exist and are to be subject to differing post-separation treatment and/or analysis.

Such an apparatus can optionally can have diverter valves located at both the inputs and outlets of each separation channel, such that the two separation devices can be run in parallel as well as in series as described above. Such an apparatus could use a diverter valve or sample splitting device to split the sample into two (or more) separate fluidic streams, each of which enters its own separation channel. Such a set-up is advantageous in that it allows for increasing the throughput; additionally, it may be desirable to separate the sample for introduction into two or more distinct treatment and/or detection steps downstream from the apparatus. Optionally, the separation chambers can be arranged to run two different samples simultaneously, that is to say, can be run as two distinct electrophoretic devices at the same time. It will of course be recognized by those skilled in the art, given the benefit of the present disclosure, that the output from any of the above embodiments can be further directed into an apparatus in accordance with any of the above devices and apparatuses, such that, for example, a system could be established using any combination of the above devices along with the appropriate number and location of fluidic pathways or connections and diverter valves or manifolds to achieve any desired degree of separation and subsequent sample handling.

In certain preferred embodiments, the electrode chamber does not contain electrolyte, e.g., buffer. The electrodes are separated from the separation chamber by a barrier that can comprise the porous conductive membrane as described above, or optionally may comprise a conductive, substantially non-porous material, such as, for example, a ceramic material. Other suitable barrier materials will be readily apparent to one skilled in the art, given the benefit of the present disclosure. In such an embodiment, the shape of the electrode chamber would not serve to create a gradient in an otherwise uniform electric field. In certain preferred embodiments, an electrode array or a shaped resister is utilized to create the gradient in the electric field. Suitable electrode arrays are described, for example, in U.S. Pat. No. 6,277,258, hereby incorporated in its entirety for all purposes. In yet other preferred embodiments, the membrane or barrier is shaped, for example, is varied in thickness, to create the gradient in the electric field. Other suitable means for generating a gradient in the electric field will be readily apparent to one skilled in the art, given the benefit of the present disclosure.

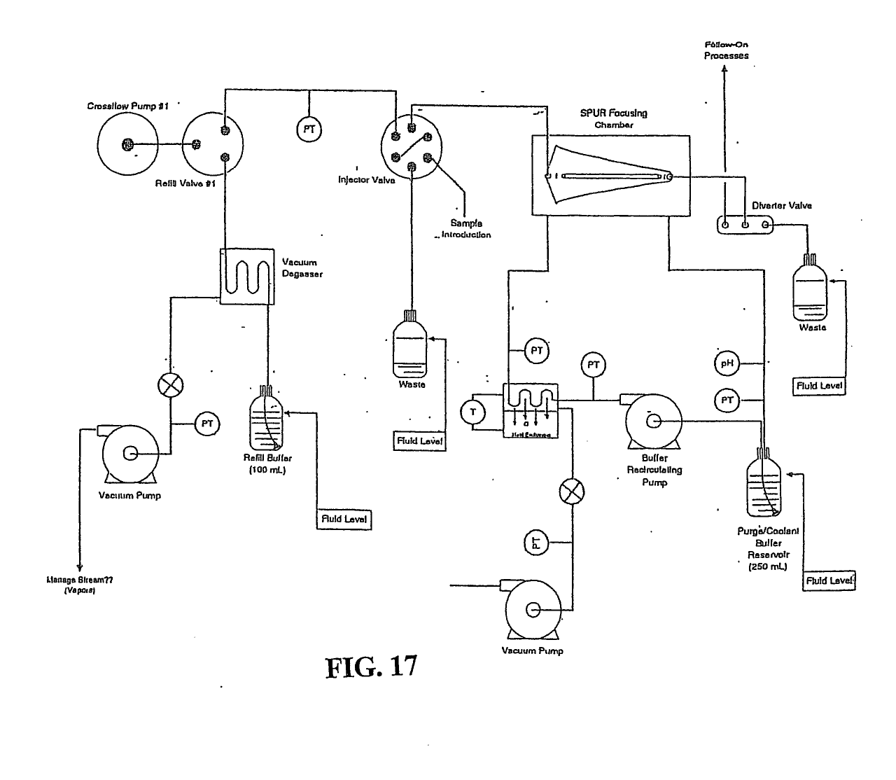

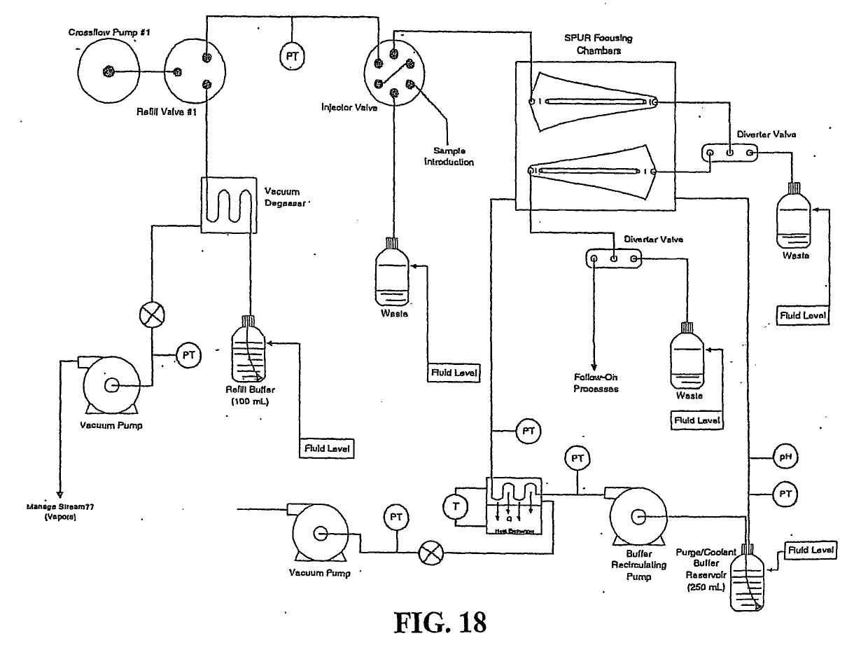

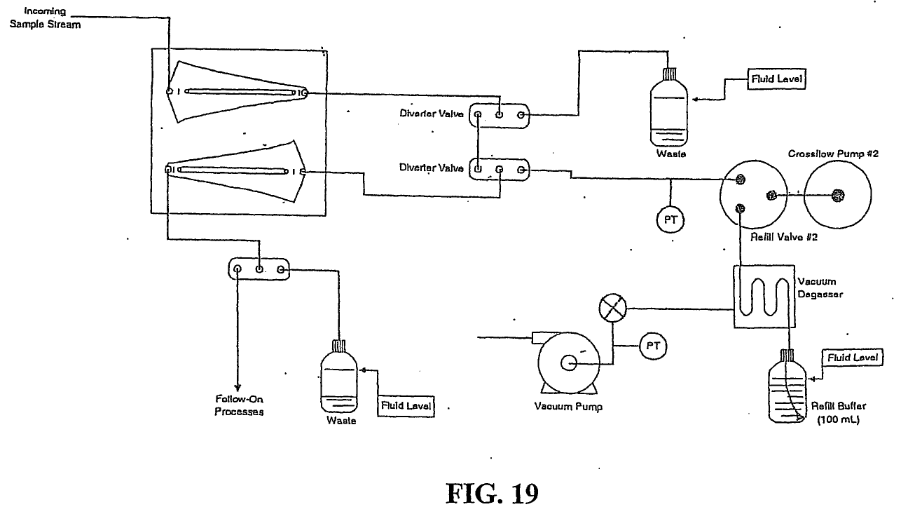

FIGS. 17, 18 and 19 are schematic illustrations showing exemplary apparatus, with FIG. 17 representing a single electrophoretic device and FIGS. 18 and 19 representing apparatuses containing two electrophoretic devices. The devices can be controlled via RS-232, LAN, or contact closure interfaces and advantageously can be used with commercially available liquid handlers to allow unattended analyte preconcentration. Fluidic samples, for example, aqueous-phase samples, are injected into the first analytical or separation chamber with the trapping electric field turned on, and the sample is allowed to separate and focus into one or more bands of focused analyte. The electric field is then lowered to allow a low mobility band of analyte to exit or be eluted from the chamber. The eluted band then passes to a follow-on process or processes as exemplified in FIG. 17, or is flowed into the second electrophoretic chamber via a diverter valve as exemplified in FIGS. 18 and 19. As described above, bands can be located within either chamber by separately manipulating the strength of the electric field in each chamber. Such a design is advantageous in that it permits the removal of bands of intermediate mobility while allowing the apparatus to retain bands of higher and lower mobility in the electrophoretic chambers. For example, desired bands of low mobility can be eluted from the first chamber and permitted to flow into the second chamber and then to a follow-on process or processes as desired. Typical operating parameters of an apparatus of this type with a 1-inch chamber are as described in Table 1 above. Advantageously, the dual-chamber apparatus further comprises a second crossflow pump, such as that found in the apparatus of FIG. 19, to provide flow through the second (downstream) electrophoretic device while the diverter valve separating the first (upstream) device from the second device is open to waste. The apparatus illustrated in FIG. 19 further provides for parallel operation of the pair of electrophoretic chambers, such that a sample can be split previous to injection and injected simultaneously onto each of the electrophoretic devices by appropriate manipulation of the pair of diverter valves. A second sample introduction site, for example, located downstream of refill valve #2, would permit the introduction of sample onto the second electrophoresis device, while an additional diverter valve located between the first diverter valve following the first device and the waste would allow diversion of desired bands to follow-on processes in a fashion similar to that found after the electrophoretic chamber of the single-device apparatus of FIG. 17. Further, such a configuration would allow the simultaneous treatment of two different samples, which need not be related. Such would effectively double the output of the apparatus by effectively allowing it to function as two distinct apparatuses. Other suitable configurations will be readily apparent to those of skill in the art, given the benefit of this disclosure.

As exemplified in FIGS. 17 and 18, certain preferred embodiments utilize a buffer recirculating pump, a vacuum pump and a heat exchanger to circulate buffer through the electrode chambers of each electrophoretic device. In this way, the buffer serves to transmit the electric field gradient to the separation chambers and to remove heat and gas generated by the electrodes. The actual connections to the electrode chambers are omitted for clarity purposes. Such a configuration would also typically be present in the apparatus illustrated in FIG. 19, and is omitted from FIG. 19 for clarity purposes. Other suitable electrode chamber fluid handling systems will be readily apparent to those skilled in the art, given the benefit of this disclosure.

In each of FIGS. 17, 18 and 19, sample is introduced via an injector valve. The samples may be directly injected, or in certain preferred embodiments may be brought into the injector valve directly or indirectly from the output of a preceding instrument, such as, for example, and HPLC instrument. In this fashion, the apparatus can be used to link up separate instruments in a hyphenated fashion, whereby the sample flows directly from one instrument into the apparatus and then into the follow-on instrument. Other suitable injection devices, for example, sample loops, etc., will be readily apparent to those skilled in the art given the benefit of the present disclosure.

Additional sample injections may be used in certain preferred embodiments to accumulate or concentrate low abundance materials while holding previous samples in either the first or, where one is present, the second chamber. Alternatively, continuous flow of sample may be so used, or a combination of continuous flow and additional injections. Further, additional such electrophoretic devices may be used in serial or in parallel networks to provide additional separation flexibility for accumulating multiple analytes for collection or analysis. Additional peripherals may be added for a specific test Other suitable apparatus designs will be readily apparent to those of skill in the art, given the benefit of this disclosure.

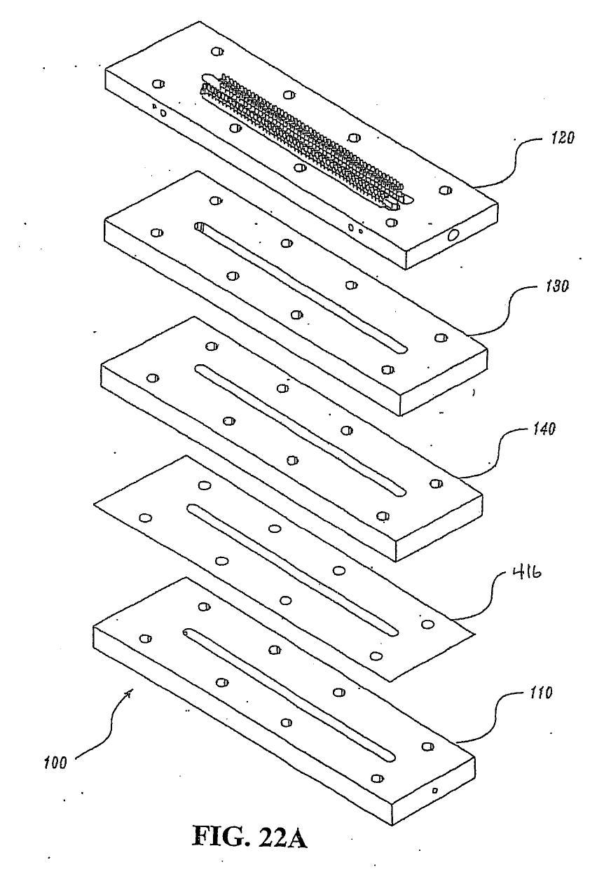

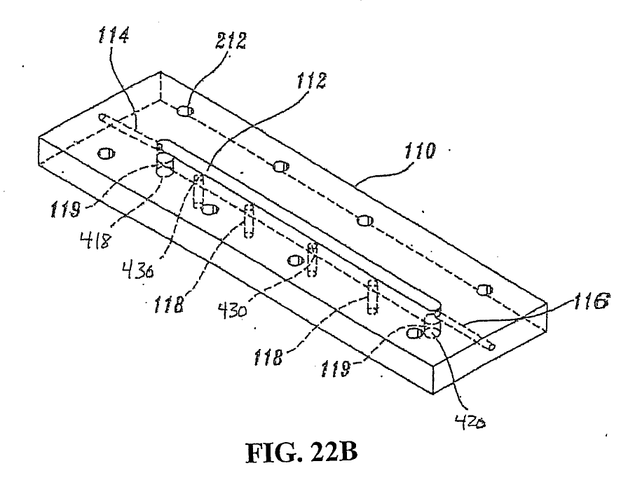

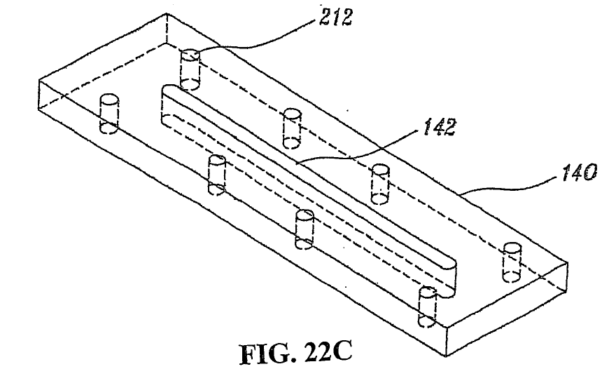

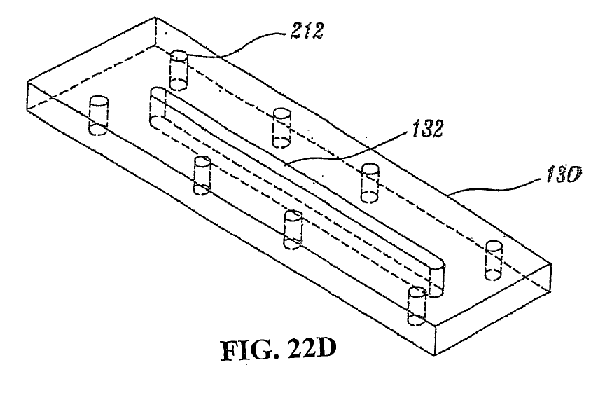

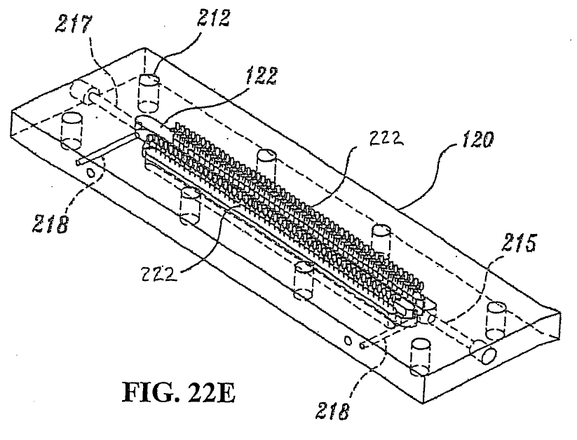

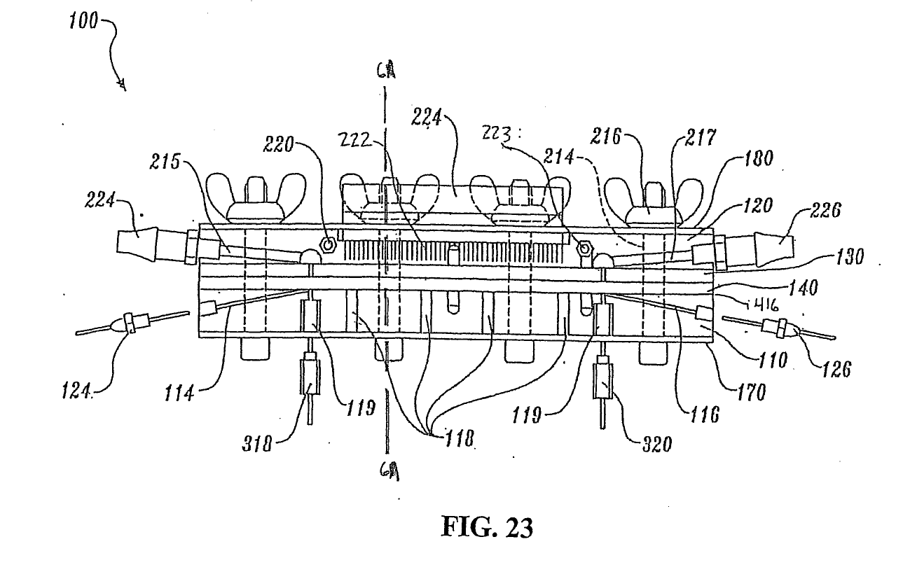

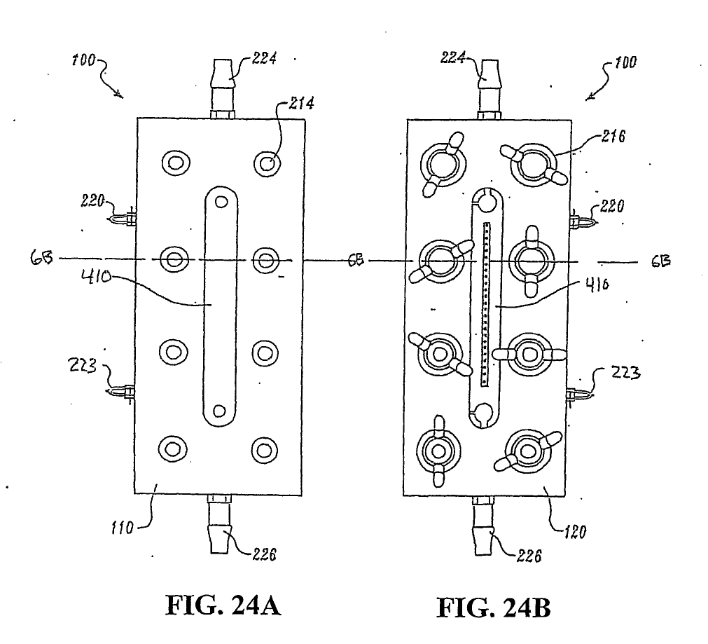

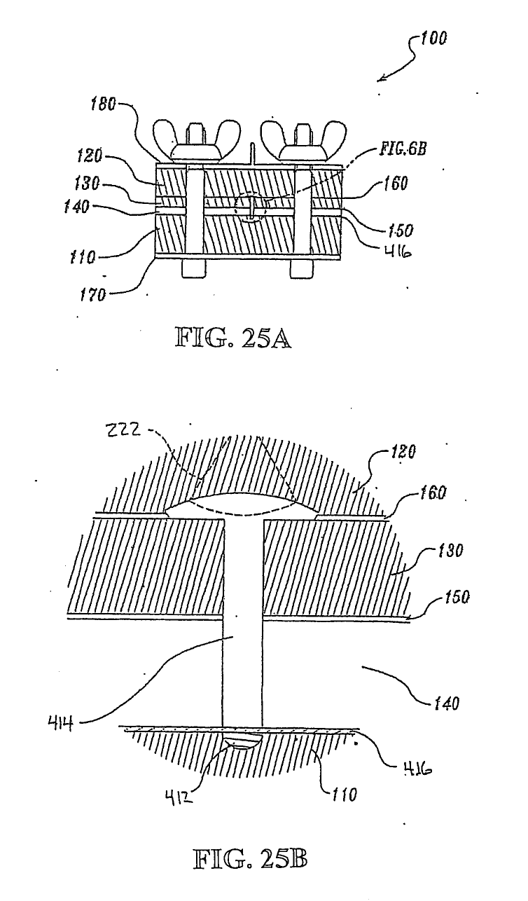

A representative electrophoretic device including a focusing chamber as described above is shown in FIGS. 22-25. FIG. 22A shows an exploded view of the device including front and rear portions. An elevation view of the device is shown in FIG. 23, and forward and rear plan views of the device as illustrated in FIGS. 24A and 24B, respectively. A cross-sectional view of a portion of a representative device illustrating the separation chamber, permeable membrane, and electrode chamber is shown in FIG. 25.

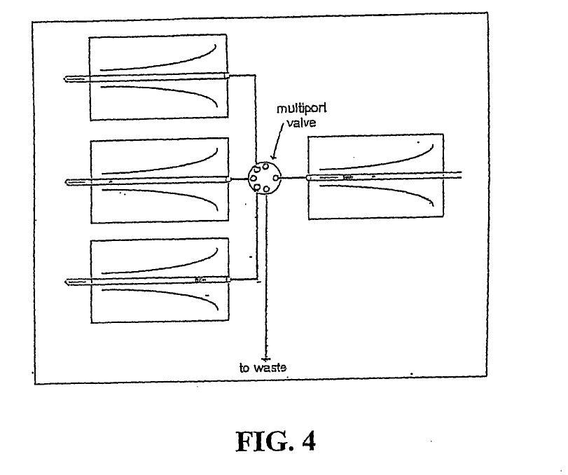

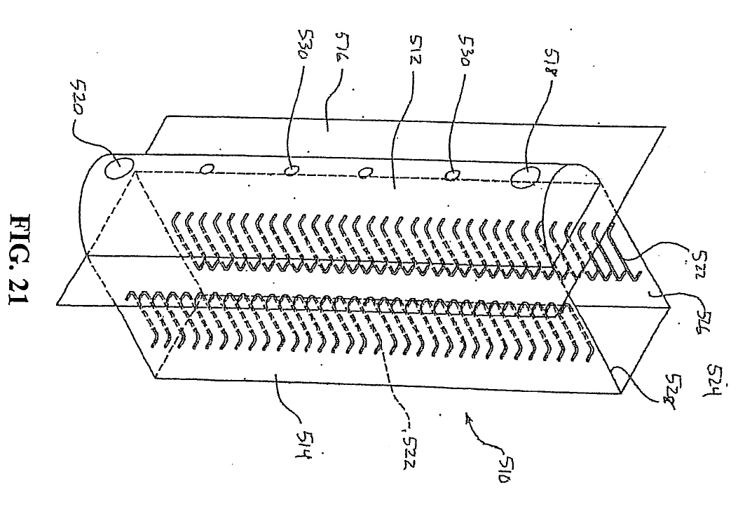

A representative device including a focusing chamber is shown in FIG. 22. The embodiment illustrated in FIG. 22 includes side-by-side electrode arrays as shown in FIG. 21. Referring to FIG. 22, device 100 has basic components including first block 110 and second block 120 separated by intermediate sheets 130 and 140. Permeable member 416 is intermediate block 110 and sheet 140. Blocks 110 and 120 and intermediate sheets 130 and 140 are formed from machinable materials. Preferably, blocks 110 and 120 and intermediate sheet 130 are formed from PLEXIGLAS and sheet 140 is formed from TEFLON. In one embodiment, each component includes a plurality of apertures 212 that are coincident with the apertures of the other components when the components are assembled. Apertures 212 receive bolts 214 (see FIG. 23) for securing the assembled components and assist in sealing the assembly. As shown in FIG. 4, the components are secured through tightening nuts 216 on bolts 214.

To form the focusing chamber, first block 110 and second block 120 include troughs 112 and 122, respectively. Trough 122 includes the electrode arrays, each array comprising a plurality of electrodes 222. Sheets 130 and 140 include apertures 132 and 142, respectively. When the components are assembled, troughs 112 and 122 and apertures 132 and 142 are coincident and form a portion of the focusing chamber 410. Intermediate sheet 140 and block 110 is permeable member 416 which divides chamber 410 into separation chamber 412 and electrode chamber 414.

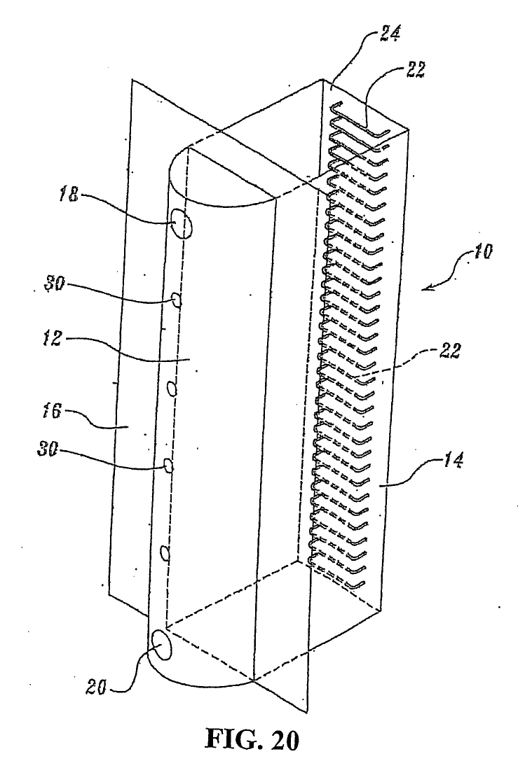

First block 110 includes conduits 114 and 116 which terminate in opposing ends of trough 112. Conduits 114 and 116 serve as inlet and outlet, respectively, for introducing a first liquid to and for removing the first liquid from the separation chamber. First block 110 further includes channels 118 which terminate in trough 112, which provide for eluting focused analytes from the device through offtake ports 30 (see FIGS. 20 and 21). Channels 119 also terminate in trough 112 and provide for introducing charged analyte and eluant to the separation chamber through inlet 418 and exiting eluant through outlet 420.

Second block 120 includes conduits 215 and 217, which terminate in opposing ends of trough 122. These conduits serve to introduce and exit liquid flow (e.g., coolant) through the electrode chamber. For embodiments of the device that include an electrode pair in addition to the electrode array, second block 120 further includes channels 218 which terminate in trough 122. Channels 218 receive electrodes 220 and 223, which like the electrode array, are in electrical communication with liquid in the electrode chamber when the device is in operation.

The assembled device is illustrated in FIGS. 23 and 24. Referring to FIG. 23, device 100 includes blocks 110 and 120 and sheets 130 and 140, and permeable member 16. Conduits 114, 116, 215, and 217, noted above, are illustrated along with connecting devices 124, 126, 224, and 226, respectively, which serve to connect the focusing chamber with its respective supplies. Inlet connection device 318 and outlet connecting device 320 are illustrated and communicate with channels 119 and separation chamber inlet 418 and outlet 420, respectively. Connector 224 leads to the device's controller and provides current to the electrode array. The representative device further includes first and second plates 170 and 180, respectively, which overlie the outward surfaces of blocks 110 and 120, respectively. Plates 170 and 180 can reinforce the assembly. Plates 170 and 180 are preferably steel plates.

FIGS. 25A and 25B are cross-sectional views of a portion of the representative device described above, taken through line 6A-6A in FIG. 23 and through line 6B-6B in FIG. 24. Referring to FIG. 25B, device 100 includes blocks 110 and 120 and sheets 130 and 140. Intermediate block 110 and sheet 140 is permeable material 416 which divides the focusing chamber into separation chamber 412 and electrode chamber 414. Sheet 140 serves as a spacer for adjusting the depth of electrode chamber 414 and, accordingly, the thickness of sheet 140 can be varied as desired. Sheet 140 is a resilient sheet and also serves to seal block 110 to the remaining components of the assembly.

Intermediate sheet 140 and sheet 130 is sealant layer 150. Sealant layer 150 includes a sealant that effectively joins sheet 140 to sheet 130 and prevents liquid from escaping the electrode chamber. Intermediate block 120 and sheet 130 is adhesive layer 160. Adhesive layer 160 includes an adhesive that effectively joins sheet 130 to block 120.

Although the above examples illustrate the use of linear electric field gradients, the software can be modified to allow point-by-point adjustment of the field including reversing the field to aid in elution of fractionated bands, isolating and mobilizing a single protein band, or stepping the gradient to improve processing capacity. In addition, because the electronic controller and the technique are largely independent of chamber capacity, there is no reason it cannot be applied at larger or smaller scales.

The dynamic electric field gradient focusing provided by the methods and devices optionally relies in part on field gradient control, which includes hardware and software. Representative gradient control hardware and software are discussed below.

A representative device including a focusing chamber was formed from two blocks of 15×6×1.2 cm³ PLEXIGLAS and a 0.3 cm thick TEFLON spacer. The front block, which houses the separation chamber (i.e., separation column or electrochromatography column), has a trough 8×0.1×0.05 cm³ machined into it, the rear block, which houses 50 controllable electrodes, has a trough 6.4×0.3×1.5 cm³, and the spacer has a 6.5×0.2 cm² slot machined through it. The trough in the front block is isolated from the spacer by the permeable material. The rear trough and slot admit a recirculating buffer that can have the same composition as the running (i.e., elution) buffer, acts both as coolant, analyte, or cathalyte, and removes electrolysis products from electrode array. Because the coolant is in contact with the separation column via a permeable material, the coolant can also be used to dialyze the running buffer to exchange salts or other low molecular weight analytes. The coolant inlet and outlet are shown in FIGS. 23 and 24.

Outside of the focusing chamber, the coolant buffer is circulated through a glass heat-exchange reservoir submerged in an ice bath. From here the coolant is introduced into the bottom of the focusing chamber and is passed over the electrodes at about 15 mL/s using a centrifugal pump (Cole-Parmer). A syringe pump controls the flow of the running buffer through the chamber at 15-150 μL/h The running buffer enters the chamber in the upper flow inlet on the front face and exits from the lower flow outlet on the front face. All lines are PEEK with flangeless fittings; sample is loaded through a 10-μL loop on a six-port injection valve (Upchurch).

The 50 chamber electrodes are made from 0.25-mm-o.d. platinum wire (Aldrich Chemical), mounted in the rear PLEXIGLAS block with a 0.05-in. pitch, and are connected to a SCSI ribbon cable using SMS-series microstrips (Samtec). Each of the SCSI leads is connected to its own printed-circuit (PC) monitor/controller board mounted on the wire wrap motherboard. Each monitor/controller board is segregated into three areas: high voltage, monitoring, and control. The high-voltage area isolates the chamber electrode voltages, which can be as high as 600 V, from the relatively sensitive electronics used to measure and adjust the electrode voltages. The monitor area of each PC board scales down the electrode voltage by about 100× and sends this signal to a commercial thermocouple board which digitizes the signal before sending it to the computer. The computer scans all 50 electrodes, compares these readings with the programmed profile, and sends a digital signal to a set of 50 DACs which tell the optical isolators to adjust the effective resistance of high-voltage line to reduce the departure of the measured electrode voltages from the programmed voltage profile. A complete scan/control cycle of the 50 controllers is taken every second. Each of the 50 controllers is mounted vertically on a wire-wrapped motherboard; power to the controllers' motherboard is drawn from the computer. A 600-V power supply (Xantrex) provides current to the column's 50 high-voltage electrodes via the 50 voltage controllers.

The device is operated as follows. After the recirculating coolant has reached operating temperature and the separating chamber has been cleaned, e.g., with 7 M urea, and equilibrated with running buffer, 10 μL of sample is injected into the chamber using a standard sample loop. Before analyte reaches the outlet, the controller is booted using a default voltage pattern and the power supply is brought up to a voltage in the range 200-600 V. The operator then selects the initial electric field gradient, and the computer program adjusts the electrode voltages until this gradient is attained, typically less than 5 min. from a “cold” start.

Where the electrophoretic mobilities or charge to mass ratios of two analytes are sufficiently close, the electric field gradient alone may be insufficient to separate them. Without wishing to be bound to any theory, it is currently understood that analytes are separated by the methods and devices disclosed here on the basis of their molecular weights by effectively applying different hydrodynamic forces to differently-sized molecules; that is to say, due to the sieving affect of the molecular sieve, molecules of different sizes effectively are subject to different hydrodynamic forces for a given flow rate of first liquid.

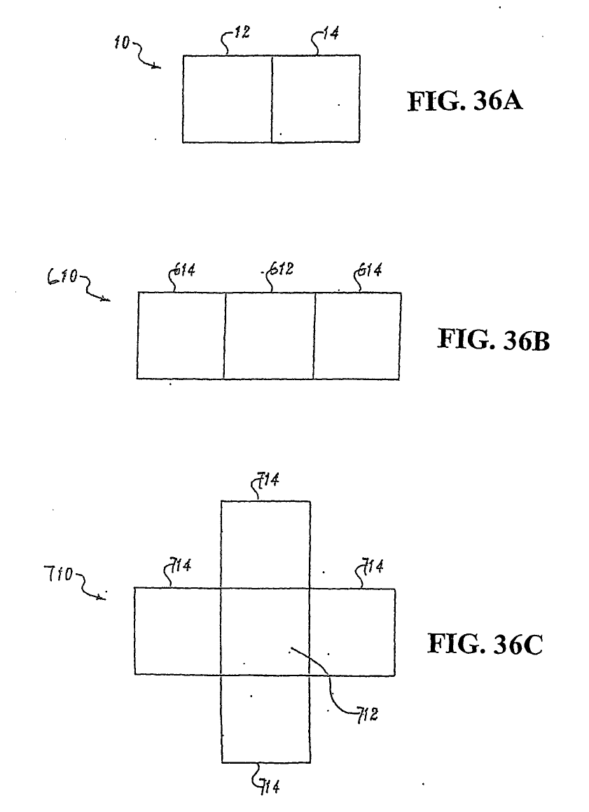

As noted above, in certain preferred embodiments the electrode chamber can include more than one electrode array. For example, two electrode arrays can be associated with a single separation chamber in a configuration in which the separation chamber is positioned in between the two arrays. Similarly, the electrode chamber can include, for example, four arrays positioned about a separation chamber in a quadrupole-type configuration. Other preferred embodiments can include more than one electrode chamber, each having one or more electrode arrays. Representative devices including one, two, and four electrode arrays are illustrated schematically in FIGS. 36A-C. Referring to FIG. 36, representative device 10 including a single electrode array (i.e., electrode chamber 14) and a separation chamber (i.e., chamber 12) is shown in FIG. 36A. FIGS. 36B and 36C illustrate representative devices 610 and 710 having two and four electrode arrays and electrode chambers 614 and 714 arranged about separation chamber 612 and 712.

While various preferred embodiments of the methods and devices have been illustrated and described, it will be appreciated that various modifications and additions can be made to such embodiments without departing from the spirit and scope of the methods and devices as defined by the following claims.

1. An electrophoresis device for focusing a charged analyte comprising:
a separation chamber having an inlet port and an outlet port defining between them a fluid flow path through the separation chamber for sample fluid comprising an analyte;
electrodes separated from the separation chamber by a membrane and operative when energized to generate an electric field gradient in the separation chamber; and
molecular sieve in the separation chamber operative to shift the location at which a stationary focused band of analyte forms under a given set of focusing process parameters including, at least
the electric field gradient, and
hydrodynamic force of sample fluid flow along the flow path through the separation chamber.

2. The electrophoresis device of claim 1 wherein there is a gradient in the electric field.

3. The electrophoresis device of claim 1 further comprising an electrode chamber with the electrodes positioned in the electrode chamber,
wherein the electrode chamber is separated from the separation chamber by the membrane, and
wherein the membrane is a permeable material.

4. The electrophoresis device of claim 3, wherein the electrode chamber is non-uniform and the separation chamber is encircled longitudinally by the electrode chamber.

5. The electrophoretic device of claim 1, wherein the electrodes comprise an electrode array.

6. The electrophoresis device of claim 5, wherein each electrode of the electrode array is capable of being individually controlled.

7. The electrophoresis device of claim 5, wherein the electrode array is operative to generate an electric field gradient which can be dynamically controlled.

8. The electrophoresis device of claim 1, wherein the degree to which the stationary focused band of charged analyte is shifted for a given set of focusing conditions varies with the molecular weight of the charged analyte.

9. The electrophoresis device of claim 1, wherein the degree to which the stationary focused band of charged analyte is shifted for a given set of focusing conditions varies with the molecular size of the charged analyte

10. The electrophoresis device of claim 1, wherein the degree to which the stationary focused band of charged analyte is shifted for a given set of focusing conditions is proportional to the molecular weight of the charged analyte.

11. The electrophoresis device of claim 1, wherein the sieve comprises a gel.

12. The electrophoresis device of claim 11, wherein the gel is an organic gel.

13. The electrophoresis device of claim 11, wherein the gel is an inorganic gel.

14. The electrophoresis device of claim 11, wherein the gel is a fixed gel.

15. The electrophoresis device of claim 11, wherein the gel is a soluble gel.

16. The electrophoresis device of claim 11, wherein the gel comprises molecules having a molecular weight of between about 2000 and about 100,000.

17. The electrophoresis device of claim 1, wherein the sieve comprises zeolite.

18. An electrophoretic device for focusing a charged analyte comprising:
a separation chamber comprising an inlet for introducing a first fluid into the separation chamber and an outlet for exiting the first liquid from the separation chamber;
an electrode chamber comprising an electrode array and an inlet for introducing a second liquid into the electrode chamber and an outlet for exiting the second liquid from the electrode chamber;
permeable material separating the separation and electrode chambers; and molecular sieve in the separation chamber operative to shift the location at which a stationary focused band of a charged analyte forms under a given set of focusing process parameters.

19. A device for focusing a charged analyte comprising:
a first block comprising a first trough having an inlet for introducing a first liquid to the first trough and an outlet for exiting the first liquid from the first trough;
a second block comprising a second trough having an inlet for introducing a second liquid to the second trough and an outlet for exiting the second liquid from the second trough, the second trough further comprising an electrode array positioned in the second trough
wherein the first trough and the second trough are substantially coincident and form a channel when the first block is sealed to the second block; and
permeable material intermediate the first and second blocks,
wherein the permeable material divides the channel formed when the first block is sealed to the second block into a first chamber and a second chamber, the second chamber including the electrode array, and wherein the first chamber comprises molecular sieve operative to shift the location at which a stationary focused band of a charged analyte forms under a given set of focusing process parameters.

20. A device for focusing a charged analyte comprising:
a first block having a first trough having an inlet for introducing a first liquid to the first trough and an outlet for exiting the first liquid from the first trough;
a second block having a second trough having an inlet for introducing a second liquid to the second trough and an outlet for exiting the second liquid from the second trough, the second trough further comprising an electrode array comprising a plurality of electrodes arranged linearly along the length of the second trough,
wherein the first trough and the second trough are substantially coincident and form a channel when the first block is sealed to the second block;
permeable material intermediate the first and second blocks;
a voltage controller for controlling the voltage applied to each electrode of the electrode array,
wherein the permeable material divides the channel formed when the first block is sealed to the second block into a first chamber and a second chamber, the second chamber including the electrode array, such that the first chamber and second chamber are in liquid communication and the first chamber is in electrical communication with the electrode array when the chambers are filled with a conductive liquid, and
wherein the first chamber comprises molecular sieve operative to shift the location at which a stationary focused band of a charged analyte forms under a given set of focusing process parameters.

21. A device for focusing and separating charged analytes comprising:
a separation chamber comprising an inlet for introducing a first liquid into the separation chamber and an outlet for exiting the first liquid from the separation chamber;
an electrode chamber comprising an electrode array and an inlet for introducing a second liquid into the electrode chamber and an outlet for exiting the second liquid from the electrode chamber;
permeable material separating the separation and electrode chambers; and
molecular sieve in the separation chamber operative to separate bands of charged analyte of similar electrophoretic mobilities and different molecular weights.

22. A device for focusing a charged analyte comprising:
a separation chamber comprising an inlet for introducing a first fluid into the separation chamber and an outlet for exiting the first liquid from the separation chamber;
an electrode array isolated from the separation chamber and operative to establish an electric field gradient in the separation chamber; and
molecular sieve in the separation chamber operative to shift the location at which a stationary focused band of a charged analyte forms under a given set of focusing process parameters.

23. An electrophoresis device for focusing a charged analyte comprising:
a separation chamber;
means for generating an electric field in the separation chamber; and
molecular sieve in the first chamber operative to shift the location at which a stationary focused band of a charged analyte forms under a given set of focusing process parameters.

24. A method for focusing a charged analyte comprising:
providing an electrophoresis device for focusing the analyte, comprising:
a separation chamber having an inlet port and an outlet port defining between them a fluid flow path through the separation chamber for sample fluid comprising the analyte;
electrodes separated from the separation chamber by a membrane and operative when energized to generate an electric field gradient in the separation chamber; and
molecular sieve in the separation chamber operative to shift the location at which a stationary focused band of the analyte forms under a given set of focusing process parameters including, at least
the electric field gradient, and
hydrodynamic force of sample fluid flow along the flow path through the separation chamber; and
introducing a flow of sample fluid into the separation chamber, the sample fluid comprising the analyte;
energizing at least a subset of the electrodes to establish an electric field gradient in the separation chamber effective to focus the analyte in the separation chamber.

24. The method of claim 24, wherein the analyte is a charged analyte.

24. The method of claim 24, wherein the analyte is focused withy the aid of lipids, micelles or vesicles in the sample fluid.

25. The method of claim 24, wherein the molecular sieve comprises a gel.

26. The method of claim 25, wherein the gel is an organic gel.

27. The method of claim 25, wherein the gel is an inorganic gel.

28. The method of claim 25, wherein the gel is a fixed gel.

29. The method of claim 25, wherein the gel is a soluble gel.

30. The method of claim 25, wherein the gel has a molecular weight of between about 2000 and about 100,000.

31. The method of claim 24, wherein the molecular sieve comprises zeolite.

32. The method of claim 24, wherein the charged analyte comprises a biological molecule.

33. The method of claim 32, wherein the charged analyte comprises DNA.

34. The method of claim 32, wherein the charged analyte comprises RNA.

35. The method of claim 32, wherein the charged analyte comprises protein.

36. The method of claim 24, wherein the charged analyte comprises a molecule sorbed to a detergent.

37. The method of claim 36, wherein the detergent comprises SDS.

38. The method of claim 24, wherein additional fluid comprising charged analyte is introduced into the first chamber and focused.

39. The method of claim 24, wherein the charged analyte comprises an uncharged material sorbed into a charged carrier.

40. The method of claim 24, wherein the electric field gradient is changed during the focusing of the analyte.

41. The method of claim 24, wherein the electric field gradient is dynamically controlled.

42. The method of claim 24, wherein the electrophoresis device further comprises an electrode chamber with the electrodes positioned proximate the electrode chamber, wherein the electrode chamber is separated from the separation chamber by the membrane, and wherein the membrane is a permeable material.

43. The method of claim 24, wherein the electrodes comprise an electrode array.

44. The method of claim 43, wherein each electrode of the electrode array is capable of being individually controlled.

45. The method of claim 43, wherein the electrode array is operative to generate an electric field gradient which can be dynamically controlled.

46. A method for focusing a charged analyte comprising:
providing a device for focusing a charged analyte comprising:
a separation chamber comprising an inlet for introducing a first liquid into the separation chamber and an outlet for exiting the first liquid from the separation chamber;
an electrode chamber comprising an electrode array and an inlet for introducing a second liquid into the electrode chamber and an outlet for exiting the second liquid from the electrode chamber; and
permeable material separating the separation and electrode chambers;
introducing a first liquid comprising at least one charged analyte into the separation chamber; and
applying an electric field gradient to the charged analyte in the first liquid to focus the charged analyte in the electric field gradient,
wherein the separation chamber contains molecular sieve operative to shift the location at which a stationary focused band of a charged analyte forms under a given set of focusing process parameters.

47. A method for focusing a charged analyte in a fluid medium comprising:
providing a device for focusing a charged analyte comprising:
a first block comprising a first trough having an inlet for introducing the first liquid to the first trough and an outlet for exiting the first liquid from the first trough;
a second block comprising a second trough having an inlet for introducing a second liquid to the second trough and an outlet for exiting the second liquid from the second trough, the second trough further comprising an electrode array positioned in the second trough,
wherein the first trough and the second trough are substantially coincident- and form a channel when the first block is sealed to the second block; and
permeable material intermediate the first and second blocks,
wherein the permeable material divides the channel formed when the first block is sealed to the second block into a first chamber and a second chamber, the second chamber including the electrode array;
introducing a first liquid comprising at least one charged analyte into the first chamber;
applying an electric field gradient to the charged analyte to cause the charged analyte to focus in a region of the first chamber,
wherein the first chamber comprises molecular sieve operative to shift the location at which a stationary focused band of charged analyte forms under a given set of focusing process parameters.

48. A method of separating charged analytes comprising:
providing a device for focusing a charged analyte comprising a separation chamber and electrodes separated from the separation chamber by a conductive membrane and operative to generate an electric field in the separation chamber; introducing a first liquid comprising at least one charged analyte into the first chamber; and
applying an electric field gradient to the plurality of charged analytes in the first liquid to focus the plurality of charged analytes in the electric field gradient into stationary bands of charged analytes, each charged analyte forming a stationary focused band,
wherein the first chamber contains molecular sieve operative to shift the location at which each stationary focused band of charged analyte forms under a given set of focusing process parameters.

49. The method of claim 48, wherein stationary focused bands of charged analyte are separated on the basis of their molecular weights.

50. The method of claim 48, wherein the plurality of charged analytes comprises charged analytes that have the same charge to mass ratio.

51. The method of claim 48, wherein the plurality of charged analytes comprises charged analytes that have the same electrophoretic mobility.

52. The method of claim 48, wherein each stationary focused band of charged analyte is focused each at a stable location separate from the other charged analytes.

53. A method of separating charged analytes comprising:
providing a device for focusing a charged analyte comprising:
a separation chamber comprising an inlet for introducing a first liquid into the separation chamber and an outlet for exiting the first liquid from the separation chamber;
an electrode chamber comprising an electrode array and an inlet for introducing a second liquid into the electrode chamber and an outlet for exiting the second liquid from the electrode chamber; and
permeable material separating the separation and electrode chambers;
introducing a first liquid comprising a plurality of charged analytes into the separation chamber, and
applying an electric field gradient to the plurality of charged analytes in the first liquid to focus the plurality of charged analytes in the electric field gradient into stationary bands of charged analytes, each charged analyte forming a stationary focused band,
wherein the separation chamber contains molecular sieve operative to shift the location at which each stationary focused band of charged analyte forms under a given set of focusing process parameters.

54. The method of claim 53, wherein the molecular sieve is operable to separate the stationary focused bands of charged analyte of similar electrophoretic mobilities and different molecular weights, each at a stable location separate from the other charged analytes.
